SEQUENCE LISTING (3 sequences)

SEQ ID NO: 1 - DNA, 954 bp, Homo sapiens
  source 1..954: mol_type = genomic DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagtgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 2 - DNA, 954 bp, Homo sapiens
  source 1..954: mol_type = genomic DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

SEQ ID NO: 3 - DNA, 954 bp, Homo sapiens
  source 1..954: mol_type = genomic DNA
atgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactga

(19) United States
(12) Patent Application Publication
(10) Pub. No.: US 2025/0334595 A1
(43) Pub. Date: Oct. 30, 2025

(54) COMPOSITION FOR USE TO SUPPRESS INHIBITION OF APOE MEASUREMENT BY LIPID AND USE THEREOF

(71) Applicants: Fujirebio Inc., Tokyo (JP); Fujirebio Europe N.V., Gent (BE)

(72) Inventors: Hisashi NOJIMA, Tokyo (JP); Katsumi AOYAGI, Tokyo (JP); Kazuyuki UMEDA, Gent (BE); Rosina DEGRIECK, Gent (BE); Ina VANDENBROUCKE, Gent (BE)

(21) Appl. No.: 19/008,409

(22) Filed: Jan. 2, 2025

(30) Foreign Application Priority Data
Jan. 31, 2024 (JP) 2024-013746

(51) Int. Cl. G01N 33/92; G01N 33/543

(52) U.S. Cl. CPC G01N 33/92; G01N 33/54326; G01N 2333/775

(57) ABSTRACT

A kit for use to measure an apolipoprotein E (ApoE), comprising: a surfactant having a steroid skeleton; and an ApoE measurement reagent.

### RELATED APPLICATION

This application claims priority from Japanese Patent Application No. 2024-013746 filed on Jan. 31, 2024. The entire disclosure of this Japanese patent application is herein incorporated by reference in its entirety.

### Sequence Listing Submission via EFS-Web

The contents of the electronic sequence listing (sequencelisting.xml; Size: 6,322 bytes; and Date of Creation: Jan. 26, 2024) is herein incorporated by reference in its entirety.

### TECHNICAL FIELD

The present disclosure relates to a composition for use to suppress inhibition of ApoE measurement by a lipid and use thereof.

### BACKGROUND OF THE INVENTION

An apolipoprotein E (ApoE) is a glycoprotein involved in transport and metabolism of lipids such as cholesterol, and a mature human ApoE protein is known to be composed of 299 amino acids. The APOE gene includes three different alleles (ε2, ε3, and ε4) encoding three different ApoE protein isoforms (ApoE2, ApoE3, and ApoE4). The three ApoE isoforms differ from one another in the 112th amino acid residue and the 158th amino acid residue. These amino acid residues are both cysteine residues (Cys112/Cys158) in ApoE2, Cys112/Arg158 in ApoE3, and Arg112/Arg158 in ApoE4. These allele combinations result in three homozygous types (E2/E2; E3/E3; and E4/E4) and three heterozygous types (E2/E3; E3/E4; E2/E4).

The APOE alleles are known to correlate with the risk of developing Alzheimer's disease (WO 1994/009155; Patent Literature 1). People carrying two APOE4 alleles (homozygous type E4/E4) have a 10 times or more higher risk of developing Alzheimer's disease (AD) than people carrying no APOE4 allele. Also, people carrying one APOE4 allele (heterozygous type) have a several times or more higher risk of developing AD than people carrying no APOE4 allele. The APOE alleles are also known to affect the age at onset of AD. Accordingly, checking the presence of the APOE4 allele is important for prediction of the risk of developing Alzheimer's disease.

Furthermore, the APOE4 allele type has been reported as a risk factor that causes a side effect, namely amyloid-related imaging abnormalities (ARIA), in patients receiving disease-modifying therapy (DMT) for AD (Cummings, J., Apostolova, L., Rabinovici, G.D. et al., “Lecanemab: Appropriate Use Recommendations.”, J. Prev. Alzheimers Dis., 2023, 10, pages 362-377; Non-Patent Literature 1). Accordingly, genotyping and phenotyping of the APOE gene are increasingly important.

A nucleic acid test is mainly used as a method for detecting the APOE4 allele. In addition, a method for checking the presence of ApoE4 by measuring the ApoE4 protein through immunoassay, and a method for checking whether or not the genotype is the homozygous E4/E4 based on a ratio between the ApoE4 protein and the ApoE protein measured through immunoassay are known as the method for detecting the APOE4 allele (Yuri T, Degrieck R, Minczakiewicz D, Sato H, Kamada J, Nakazawa T, et al. “Estimation of the allelic status of apolipoprotein E4 isoforms with fully automated LUMIPULSE (registered trademark) assays.” Explor Neurosci. 2023; 2: pages 238-244; Non-Patent Literature 2).

Patent Literature 1 is an example of related art.

Non-Patent Literature 1 and Non-Patent Literature 2 are also examples of related art.

### SUMMARY OF THE INVENTION

Thus, the inventors of the present invention measured the ApoE protein in blood samples, and found that, even when the ApoE contents were the same, the results of the ApoE protein measurement widely varied depending on the donors of the blood samples, that is, the measurement could be inhibited. Also, the inventors of the present invention found that the inhibition of the measurement (measurement interference) was caused by a lipid.

Therefore, it is an object of the present disclosure to provide a composition for use to suppress inhibition of ApoE protein measurement by a lipid.

In order to achieve the object above, a composition for use to suppress inhibition of ApoE protein measurement by a lipid according to the present disclosure contains a surfactant having a steroid skeleton.

A kit for use to measure an ApoE according to the present disclosure includes a surfactant having a steroid skeleton, and an ApoE measurement reagent.

A method for measuring an ApoE according to the present disclosure includes a measurement step of measuring an ApoE in a target sample by bringing the sample into contact with an ApoE measurement reagent in the presence of a surfactant having a steroid skeleton.

With the present disclosure, it is possible to suppress an inhibitory effect of a lipid on the ApoE measurement.

### DETAILED DESCRIPTION OF THE INVENTION

### Definitions

In this specification, the term “apolipoprotein E” (ApoE) means a protein that belongs to the family of lipid-binding proteins called apolipoproteins and mediates lipid transport between tissues. The ApoE is a mature ApoE composed of 299 amino acids that results from cleavage of a signal peptide at the N-terminus of a precursor protein composed of 317 amino acids. The APOE gene is known to include three major alleles/isoforms, namely the Apo-ε2 gene (APOE2), the Apo-ε3 gene (APOE3), and the Apo-ε4 gene (APOE4). Two polymorphisms, namely rs429358 and rs7412, define the alleles of the APOE gene. In the APOE2 gene, the codon encoding the 112th amino acid in the APOE gene is a codon (TGC) encoding Cys, and the codon encoding the 158th amino acid is a codon (TGC) encoding Cys. In the APOE3 gene, the codon encoding the 112th amino acid in the APOE gene is a codon (TGC) encoding Cys, and the codon encoding the 158th amino acid is a codon (CGC) encoding Arg. In the APOE4 gene, the codon encoding the 112th amino acid in the APOE gene is a codon (CGC) encoding Arg, and the codon encoding the 158th amino acid is a codon (CGC) encoding Arg. The base sequence encoding the precursor protein of the human APOE3 corresponds to the base sequence from position 70 to position 1023 in the base sequence of GenBank Accession Number NM_000041.4, and specific examples of the base sequence encoding the human ApoE2 to ApoE4 are the following base sequences. The underlined bases in each of the following base sequences of SEQ ID NOs: 1 to 3 are the codon encoding the 112th amino acid and the codon encoding the 158th amino acid of the mature ApoE in order from front to back.

-APOE2 gene
(SEQ ID NO: 1)
5′-ATGAAGGTTCTGTGGGCTGCGTTGCTGGTCACATTCCTGGCAGGATG
CCAGGCCAAGGTGGAGCAAGCGGTGGAGACAGAGCCGGAGCCCGAGCTGC
GCCAGCAGACCGAGTGGCAGAGCGGCCAGCGCTGGGAACTGGCACTGGGT
CGCTTTTGGGATTACCTGCGCTGGGTGCAGACACTGTCTGAGCAGGTGCA
GGAGGAGCTGCTCAGCTCCCAGGTCACCCAGGAACTGAGGGCGCTGATGG
ACGAGACCATGAAGGAGTTGAAGGCCTACAAATCGGAACTGGAGGAACAA
CTGACCCCGGTGGCGGAGGAGACGCGGGCACGGCTGTCCAAGGAGCTGCA
GGCGGCGCAGGCCCGGCTGGGCGCGGACATGGAGGACGTGTGCGGCCGCC
TGGTGCAGTACCGCGGCGAGGTGCAGGCCATGCTCGGCCAGAGCACCGAG
GAGCTGCGGGTGCGCCTCGCCTCCCACCTGCGCAAGCTGCGTAAGCGGCT
CCTCCGCGATGCCGATGACCTGCAGAAGTGCCTGGCAGTGTACCAGGCCG
GGGCCCGCGAGGGCGCCGAGCGCGGCCTCAGCGCCATCCGCGAGCGCCTG
GGGCCCCTGGTGGAACAGGGCCGCGTGCGGGCCGCCACTGTGGGCTCCCT
GGCCGGCCAGCCGCTACAGGAGCGGGCCCAGGCCTGGGGCGAGCGGCTGC
GCGCGCGGATGGAGGAGATGGGCAGCCGGACCCGCGACCGCCTGGACGAG
GTGAAGGAGCAGGTGGCGGAGGTGCGCGCCAAGCTGGAGGAGCAGGCCCA
GCAGATACGCCTGCAGGCCGAGGCCTTCCAGGCCCGCCTCAAGAGCTGGT
TCGAGCCCCTGGTGGAAGACATGCAGCGCCAGTGGGCCGGGCTGGTGGAG
AAGGTGCAGGCTGCCGTGGGCACCAGCGCCGCCCCTGTGCCCAGCGACAA
TCACTGA-3′
-APOE3 gene
(SEQ ID NO: 2)
5′-ATGAAGGTTCTGTGGGCTGCGTTGCTGGTCACATTCCTGGCAGGATG
CCAGGCCAAGGTGGAGCAAGCGGTGGAGACAGAGCCGGAGCCCGAGCTGC
GCCAGCAGACCGAGTGGCAGAGCGGCCAGCGCTGGGAACTGGCACTGGGT
CGCTTTTGGGATTACCTGCGCTGGGTGCAGACACTGTCTGAGCAGGTGCA
GGAGGAGCTGCTCAGCTCCCAGGTCACCCAGGAACTGAGGGCGCTGATGG
ACGAGACCATGAAGGAGTTGAAGGCCTACAAATCGGAACTGGAGGAACAA
CTGACCCCGGTGGCGGAGGAGACGCGGGCACGGCTGTCCAAGGAGCTGCA
GGCGGCGCAGGCCCGGCTGGGCGCGGACATGGAGGACGTGTGCGGCCGCC
TGGTGCAGTACCGCGGCGAGGTGCAGGCCATGCTCGGCCAGAGCACCGAG
GAGCTGCGGGTGCGCCTCGCCTCCCACCTGCGCAAGCTGCGTAAGCGGCT
CCTCCGCGATGCCGATGACCTGCAGAAGCGCCTGGCAGTGTACCAGGCCG
GGGCCCGCGAGGGCGCCGAGCGCGGCCTCAGCGCCATCCGCGAGCGCCTG
GGGCCCCTGGTGGAACAGGGCCGCGTGCGGGCCGCCACTGTGGGCTCCCT
GGCCGGCCAGCCGCTACAGGAGCGGGCCCAGGCCTGGGGCGAGCGGCTGC
GCGCGCGGATGGAGGAGATGGGCAGCCGGACCCGCGACCGCCTGGACGAG
GTGAAGGAGCAGGTGGCGGAGGTGCGCGCCAAGCTGGAGGAGCAGGCCCA
GCAGATACGCCTGCAGGCCGAGGCCTTCCAGGCCCGCCTCAAGAGCTGGT
TCGAGCCCCTGGTGGAAGACATGCAGCGCCAGTGGGCCGGGCTGGTGGAG
AAGGTGCAGGCTGCCGTGGGCACCAGCGCCGCCCCTGTGCCCAGCGACAA
TCACTGA-3′
-APOE4 gene
(SEQ ID NO: 3)
5′-ATGAAGGTTCTGTGGGCTGCGTTGCTGGTCACATTCCTGGCAGGATG
CCAGGCCAAGGTGGAGCAAGCGGTGGAGACAGAGCCGGAGCCCGAGCTGC
GCCAGCAGACCGAGTGGCAGAGCGGCCAGCGCTGGGAACTGGCACTGGGT
CGCTTTTGGGATTACCTGCGCTGGGTGCAGACACTGTCTGAGCAGGTGCA
GGAGGAGCTGCTCAGCTCCCAGGTCACCCAGGAACTGAGGGCGCTGATGG
ACGAGACCATGAAGGAGTTGAAGGCCTACAAATCGGAACTGGAGGAACAA
CTGACCCCGGTGGCGGAGGAGACGCGGGCACGGCTGTCCAAGGAGCTGCA
GGCGGCGCAGGCCCGGCTGGGCGCGGACATGGAGGACGTGCGCGGCCGCC
TGGTGCAGTACCGCGGCGAGGTGCAGGCCATGCTCGGCCAGAGCACCGAG
GAGCTGCGGGTGCGCCTCGCCTCCCACCTGCGCAAGCTGCGTAAGCGGCT
CCTCCGCGATGCCGATGACCTGCAGAAGCGCCTGGCAGTGTACCAGGCCG
GGGCCCGCGAGGGCGCCGAGCGCGGCCTCAGCGCCATCCGCGAGCGCCTG
GGGCCCCTGGTGGAACAGGGCCGCGTGCGGGCCGCCACTGTGGGCTCCCT
GGCCGGCCAGCCGCTACAGGAGCGGGCCCAGGCCTGGGGCGAGCGGCTGC
GCGCGCGGATGGAGGAGATGGGCAGCCGGACCCGCGACCGCCTGGACGAG
GTGAAGGAGCAGGTGGCGGAGGTGCGCGCCAAGCTGGAGGAGCAGGCCCA
GCAGATACGCCTGCAGGCCGAGGCCTTCCAGGCCCGCCTCAAGAGCTGGT
TCGAGCCCCTGGTGGAAGACATGCAGCGCCAGTGGGCCGGGCTGGTGGAG
AAGGTGCAGGCTGCCGTGGGCACCAGCGCCGCCCCTGTGCCCAGCGACAA
TCACTGA-3′

In this specification, the term “target” means an animal or a cell, tissue, or organ derived from an animal, and is particularly used to mean a human. The term “animal” means a human and non-human animals. Examples of the non-human animals include mammals such as a mouse, a rat, a hamster, a rabbit, a goat, a cow, a horse, a dog, a cat, a pig, a monkey, a dolphin, and a sea lion.

In this specification, a “sample” may be a sample that contains an ApoE or a sample that can contain an ApoE. Examples of the sample include biological samples such as organismic samples and specimens. Examples of the biological samples include samples containing a body fluid, a cell, a tissue, an organ, and the like, and specific examples thereof include samples such as feces, whole blood, serum, plasma, cerebrospinal fluid, puncture fluid, and bile. The biological samples are preferably blood samples such as whole blood, serum, and plasma. The sample may be a liquid sample or a solid sample. When the sample is a solid sample, it is preferable to prepare a liquid sample by mixing the solid sample with a liquid in the present disclosure. Examples of the liquid include water, a physiological saline solution; and buffer solutions such as a Hank's buffer solution, Good's buffer solutions (e.g., HEPES buffer solution, tricine buffer solution, and the like), a Tris buffer solution, a phosphate buffer solution, and a glycine buffer solution.

In this specification, the term “kit” means a unit whose constituent elements (e.g., an inspection reagent, a diagnostic reagent, a test reagent, a label, a substrate, and an operation manual) to be provided are generally divided into two or more sections and are provided in that manner. The kit is favorably used to, for example, provide compositions that are preferably mixed just before use rather than being provided as a mixture in consideration of stability. The kit preferably includes an instruction manual or operation manual relating to a way to use provided constituent elements (e.g., an inspection reagent, a diagnostic reagent, a test reagent, and the like), or an instruction manual or operation manual that describes treatment of the components. When the kit is used as a reagent kit in this specification, the kit may include an instruction manual or the like that describes a way to use an inspection reagent, a diagnostic reagent, and the like.

In this specification, the “instruction manual” or “operation manual” describes a way to use the present disclosure for medical doctors or other users. The instruction manual describes, for example, instructions relating to a suppression method or measurement method of the present disclosure, or to a way to use a composition or kit of the present disclosure. This instruction manual is prepared in accordance with the form prescribed by the competent authority (e.g., the Ministry of Health, Labour and Welfare in Japan, the Food and Drug Administration (FDA) in the U.S., and the European Medicines Agency (EMA) in Europe) in a country where the present disclosure is implemented, and states clearly that the present disclosure is approved by the competent authority. The instruction manual may be what is called a package insert, and is generally provided as a print medium, but there is no limitation thereto. For example, the instruction manual may also be provided in the form of an electronic medium (e.g., a web site provided on the Internet or an e-mail) or the like.

In this specification, the term “gene” refers to a factor that determines an inherited trait, and the “gene” may refer to a “polynucleotide”, an “oligonucleotide”, and a “nucleic acid”.

In this specification, the term “protein”, “peptide”, or “polypeptide” means a polymer composed of unmodified amino acids (natural amino acids), modified amino acids, and/or artificial amino acids. The polypeptide is, for example, a peptide composed of 10 or more amino acids.

In this specification, the term “nucleic acid”, “polynucleotide”, or “oligonucleotide” means a polymer of deoxyribonucleotides (DNAs), ribonucleotides (RNAs), and/or modified nucleotides. The nucleic acid may be a single-stranded nucleic acid molecule or a double-stranded nucleic acid molecule. The polynucleotide may be composed of natural nucleotides or modified or artificial nucleotides, or both of these types of nucleotides.

In this specification, the term “label” means a substance for distinguishing a target molecule or substance from the other molecules or substances. Examples of the label include fluorescent labels such as fluorescent dyes and fluorescent substances (e.g., fluorescein, fluorescein isothiocyanate, and rhodamine); chemiluminescent labels such as luciferin and aequorin; luminescent substances such as acridinium derivatives; enzyme labels such as horseradish peroxidase, alkaline phosphatase, β-galactosidase (β-gal), glucose oxidase, and luciferase; and radioisotope (RI) labels such as ³H, ¹⁴C, ³²P, ³⁵S, and ¹²⁵I.

In this specification, a “binding molecule” is a molecule capable of binding to a predetermined molecule. Examples of the binding molecule include nucleic acid molecules, proteins, sugar chains, and the like that are capable of binding to the predetermined molecule. Specific examples of the binding molecule include aptamers, antibodies, receptors, and ligands that are capable of binding to the predetermined molecule. The binding molecule may be, for example, a known binding molecule capable of binding to the predetermined molecule, or a binding molecule that is newly prepared using the SELEX method, the phage display method, or the like. The term “antibody” means a protein that includes one or more polypeptides each substantially or partially encoded by an immunoglobulin gene or immunoglobulin gene fragment. Examples of the antibody include polyclonal antibodies and monoclonal antibodies. Examples of the isotypes of the antibody include IgG (e.g., IgG1, IgG2, IgG3, IgG4, and the like), IgM, IgA (e.g., IgA1, IgA2, and the like), IgE, IgD, and IgY. Examples of the origin of the antibody include animals such as mammals (e.g., a mouse, a rat, a hamster, a rabbit, a goat, a cow, a horse, a camel, and an alpaca); birds (e.g., a chicken and an ostrich); and cartilaginous fishes (e.g., a shark). The antibody may be, for example, a heavy chain antibody (VHH antibody) derived from a Camelidae animal, an immunoglobulin new antigen receptor (IgNAR) derived from a cartilaginous fish, and an antibody fragment (e.g., Fab, Fab′, F(ab′)₂, a single domain antibody (nanobody), or the like), a recombinant antibody (e.g., scFv, disulfide-bond Fv (dsFv), diabody, minibody, or the like). The antibody may be an antibody-like molecule (e.g., affibody, anticalin, DARPins, monobody, or the like) produced through a molecular biological technique such as phage display and/or through a protein engineering technique using an existing protein motif.

Information on the sequences of proteins described in this specification or the sequences of nucleic acids (e.g., DNAs or RNAs) encoding the proteins is available from Protein Data Bank, UniProt, GenBank, or the like. In addition, the nucleic acid sequences of RNAs can be obtained from the base sequences of the corresponding DNAs using sequence conversion software as appropriate.

The following specifically describes examples of the present disclosure. The descriptions of the other disclosures can be applied to each disclosure below unless otherwise stated.

### Composition for Use to Suppress Inhibition of ApoE Measurement by Lipid

An aspect of the present disclosure provides a composition capable of suppressing inhibition of apolipoprotein E (ApoE) measurement by a lipid. The composition for use to suppress inhibition of apolipoprotein E (ApoE) measurement by a lipid according to the present disclosure (this composition may also be referred to as the “composition” hereinafter) contains a surfactant having a steroid skeleton.

The inventors of the present invention measured an ApoE in blood samples, and found that, even when the ApoE contents were the same, the results of the ApoE measurement widely varied depending on the blood sources. As a result of extensive research, the inventors of the present invention found that, as the lipid content in the sample increased, the ApoE measurement was inhibited, and further found that the inhibition of the ApoE measurement by a lipid could be suppressed by using a surfactant having a steroid skeleton among various types of surfactants, and thus achieved the present disclosure. With the composition of the present disclosure, the inhibition of ApoE measurement by a lipid can be suppressed, and therefore, an ApoE can be measured even when, for example, the sample contains a lipid such as cholesterol or a neutral lipid. Therefore, with the composition of the present disclosure, an ApoE can also be favorably measured in, for example, a biological sample containing the lipid.

In the present disclosure, examples of the lipid include cholesterol, a neutral fat, chylomicron, and a very low density lipoprotein (VLDL). The lipid is, for example, a lipid contained in chyle. The composition of the present disclosure can be favorably used for, for example, a sample containing the lipid at 780 FTU (Formazin Turbidity Unit) and particularly 1580 FTU or more.

In the present disclosure, the measurement target ApoE may be any one type of ApoE, two or more types of ApoEs, or all types of ApoEs (total ApoE). The measurement target is preferably a combination of the total ApoE and any one or more types of ApoEs because, for example, the alleles and the genotypes of ApoEs can be determined. Specifically, to check the genotype of the ApoB4, the measurement target ApoE is, for example, a combination of the total ApoE and the ApoE4.

The surfactant having a steroid skeleton is a compound having a steroid skeleton as an independent cyclic structure, that is, a steroid skeleton that is not fused with another ring, or a salt thereof. The conformation of the surfactant having a steroid skeleton at position 5 may be the α conformation or the β conformation. The surfactant having a steroid skeleton may have a steroid skeleton that does not have a hydroxyl group at position 7 or a steroid skeleton that has a hydroxyl group at position 7.

The surfactant having a steroid skeleton may be, for example, a compound having a hydrophilic moiety and a steroid skeleton as a hydrophobic moiety, or a salt thereof. Examples of the hydrophilic moiety include an anionic moiety such as a sulfonate (—SO₃—), a carboxylate (—COO—), or a phosphonate (—POO₂—); a cationic moiety such as a quaternary ammonium or quaternary phosphonium that may have undergone substitution with 1 to 4 hydrocarbon groups; a nonionic hydrophilic moiety composed of a plurality of ether moieties and the like; and a group such as a hydrocarbon group having a moiety above. Accordingly, the surfactant having a steroid skeleton is an anionic surfactant, a cationic surfactant, an amphoteric surfactant, or a nonionic surfactant depending on the type of hydrophilic moiety. Examples of the hydrocarbon group include a methyl group, an ethyl group, a propyl group, an iso-propyl group, a butyl group, an iso-butyl group, a sec-butyl group, a tert-butyl group, a pentyl group, a hexyl group, a heptyl group, an octyl group, a nonyl group, a decyl group, a dodecyl (lauryl) group, a tetradecyl (myristyl) group, a hexadecyl (cetyl) group, a heptadecyl group, an octadecyl (stearyl) group, a cyclopropyl group, a cyclobutyl group, a cyclopentyl group, a cyclohexyl group, a cycloheptyl group, a cyclooctyl group, a cyclononyl group, a cyclodecyl group, a phenyl group, and a naphthalenyl group. The hydrocarbon group is preferably a hydrocarbon group having 1 to 10 carbon atoms, and more preferably an alkyl group having 1 to 6 carbon atoms.

The steroid skeleton may have 1 to 6 substituents, and preferably 1, 2, 3, or 4 substituents, in addition to the hydrophilic moiety. The substituent is not particularly limited as long as the properties (e.g., hydrophobicity) of the steroid skeleton are not greatly impaired, and examples thereof include hydrocarbon groups having 1 to 10 carbon atoms; a hydroxyl group; a hydroxyl group that has undergone substitution with a hydrocarbon group having 1 to 10 carbon atoms, such as an alkyloxy group; a hydrocarbon group having 1 to 10 carbon atoms-carbonyl-oxy group, such as an alkyl-carbonyl-oxy group; an oxo group; a formyl group; a hydrocarbon group having 1 to 10 carbon atoms-oxy-carbonyl group, such as an alkyloxy-carbonyl group; halogen atoms such as a fluorine atom, a chlorine atom, a bromine atom, and an iodine atom; a cyano group; and the like.

The surfactant having a steroid skeleton is preferably bile acid or a derivative thereof, or a salt thereof. Examples of the bile acid include deoxycholic acid, chenodeoxycholic acid, ursodeoxycholic acid, hyodeoxycholic acid, cholic acid, glycocholic acid, taurocholic acid, hyocholic acid, 5α-cyprinol, lithocholic acid, and taurodeoxycholic acid. Examples of the bile acid derivative include CHAPS (3-[(3-cholamidopropyl)dimethylammonio]propanesulfonate), BIGCHAP (N,N-bis(3-D-gluconamidopropyl)cholamide), deoxy-BIGCHAP (N,N-bis(3-D-gluconamidopropyl)deoxycholamide), and CHAPSO (3-[(3-cholamidopropyl)dimethylammonio]-2-hydroxy-1-propanesulfonate).

The salt is any salt, and examples thereof include inorganic salts, organic salts, and inner salts. Examples of the inorganic salts include metallic salts, halide salts, acid addition salts, and ammonium salts. Examples of the metallic salts include: alkali metal salts such as a lithium salt, a sodium salt and a potassium salt; and alkaline-earth metal salts such as a magnesium salt and a calcium salt. Examples of halogens included in the halide salts include fluorine, bromine, chlorine, and iodine. Examples of the acid addition salts, which are inorganic salts, include salts with inorganic acid, such as a hydrochloride, a nitrate, a sulfate, a phosphate, a carbonate, a hydrogen carbonate, and a perchlorate. Examples of the organic salts include salts with organic base, such as ammonium salts, aliphatic amine salts (e.g., a trimethylamine salt, a triethylamine salt, dicyclohexylamine salt, an ethanolamine salt, a diethanolamine salt, and triethanolamine salt), aralkylamine salts (e.g., N,N-dibenzylethylenediamine), and heteroaromatic amine salts (e.g., pyridine salt, a picoline salt, a quinoline salt, and an isoquinoline); and salts with organic acid, such as an oxalate, an acetate, a propionate, a succinate, a glycolate, a lactate, a maleate, a fumarate, a tartrate, a malate, and a citrate.

The composition of the present disclosure may contain one type of surfactant having a steroid skeleton or a plurality of types of surfactants having a steroid skeleton.

The composition of the present disclosure may be in the form of a solid such as powder or granules, or in the form of a liquid such as a slurry (suspension), a jelly, or a solution.

The composition of the present disclosure can be favorably used as an inspection reagent, test reagent, or research reagent for use to, for example, measure an ApoE in a sample that contains lipids or can contain lipids. With the composition of the present disclosure, a suppression method of the present disclosure, which will be described below, can be favorably conducted.

### Kit for Use to Measure ApoE

Another aspect of the present disclosure provides a kit for use to measure an ApoE while suppressing an inhibitory effect of a lipid on the ApoE measurement. The kit for use to measure an ApoE according to the present disclosure (this kit is also referred to as the “kit” hereinafter) includes a surfactant having a steroid skeleton, and an apolipoprotein E (ApoE) measurement reagent. With the kit of the present disclosure, it is possible to suppress the inhibitory effect of a lipid on the ApoE measurement. Accordingly, the kit of the present disclosure can be favorably used to, for example, measure an ApoE in a sample even when the sample contains the lipid.

The ApoE measurement reagent is a reagent capable of specifically detecting or measuring the ApoE, and contains, for example, a first binding molecule for the ApoE. It is preferable that the first binding molecule specifically binds to the ApoE. The first binding molecule is preferably an antibody against an ApoE or an antigen-binding fragment thereof. The measurement target may be one type of ApoE or a plurality of types of ApoEs. When a plurality of types of ApoEs are to be measured, the first binding molecule may include a first binding molecule that specifically binds to a region common to the ApoEs, or a plurality of first binding molecules that specifically bind to the corresponding ApoEs. The antibody against an ApoE may be, for example, an antibody against the ApoE2, an antibody against the ApoE3, an antibody against the ApoE4, or a combination of these antibodies, or may be an antibody (anti-Pan-ApoE antibody) that binds to all of the ApoE2, the ApoE3, and the ApoE4. These antibodies against an ApoE can be produced by employing and improving the existing known methods as appropriate. For example, when the antibody against an ApoE is a monoclonal antibody, the antibody can be produced through the method for producing a monoclonal antibody using a hybridoma (Kohler & Milstein, Nature, 256:495, 1975). The antibody against an ApoE is commercially available, and a commercially available antibody can also be used.

The first binding molecule for the ApoE may be supported by a carrier. That is to say, the ApoE measurement reagent may contain a carrier that supports the first binding molecule for the ApoE. Examples of the carrier include particles such as magnetic particles and beads; membranes such as a nitrocellulose membrane; substrates made of glass, plastic, metal, and the like; plates such as a multi-well plate, among which the particles are preferable because they offer excellent handleability. The antibody may be provided in the form of being impregnated into a medium such as filter paper. The first binding molecule for the ApoE may also be configured to be supported by a carrier at the time of ApoE measurement. That is to say, in the kit of the present disclosure, the ApoE measurement reagent may also be configured such that the components thereof are individually present and then coexist at the time of measurement, and thus the first binding molecule for the ApoE is supported by the carrier. In this case, the kit of the present disclosure may also be configured such that, for example, a first component of a pair of affinity substances is bound to the first binding molecule for the ApoE, a second component of the pair of affinity substances is bound to the carrier, and then the first binding molecule for the ApoE is supported by the carrier via an affinity bond between the first component and the second component. The pair of affinity substances include the first component and the second component, and the combination of the first component and the second component is a combination of substances that specifically bind to each other and can also be referred to as, for example, a tag and a binding partner.

A conventionally known method or a method in conformity therewith can be employed as a method for immobilizing the first binding molecule on the carrier as appropriate depending on the type of the first binding molecule, and the first binding molecule may be directly or indirectly immobilized on the surface of the carrier. The direct binding method may be as follows, for example: a carrier obtained by adding an active group to the carrier described above or a carrier having an active group serving as the carrier described above is used to bind the first binding molecule to the carrier via a covalent bond formed through a reaction between the active group and the first binding molecule. The indirect immobilization method may be, for example, a method in which the pair of affinity substances described above are used. Examples of the pair of affinity substances include a combination of biotin and avidin or streptavidin, a combination of nickel and a His tag, and a combination of an epitope tag such as a flag (trademark)-tag, an HA-tag, a T7-tag, a V5-peptide-tag, and/or a Myc-tag and an antibody against the tag. Also, the first binding molecule may be supported by the carrier by binding to the carrier through an ionic bond or being adsorbed on the carrier. When the first binding molecule for the ApoE is indirectly supported by the carrier, for example, the antibody against an ApoE and the carrier may be contained in the same container or in separate containers.

The ApoE measurement reagent may also contain a second binding molecule for, for example, the ApoE or a complex of the ApoE and the first binding molecule. In this case, it is preferable that the first binding molecule and the second binding molecule can bind to, for example, different sites of the ApoE, that is, the two binding molecules can bind to the ApoE at the same time. When a plurality of types of ApoEs are to be measured, the second binding molecule may include a second binding molecule that specifically binds to a region common to the ApoEs, or a plurality of second binding molecules that specifically bind to the corresponding ApoEs.

The first binding molecule and/or the second binding molecule may have a label. In this case, detecting the label makes it possible to detect the ApoE via the first binding molecule and/or the second binding molecule. When the ApoE measurement reagent contains the first binding molecule and the second binding molecule, it is preferable that one of the first binding molecule and the second binding molecule is supported by the carrier and the other has the label. It is more preferable that, for example, the first binding molecule is supported by the carrier and the second binding molecule has the label. A conventionally known method or a method in conformity therewith can be employed as a method for introducing a label into the first binding molecule and/or the second binding molecule as appropriate depending on the types of the first binding molecule and the second binding molecule, and the label may be directly or indirectly bound thereto. Examples of the label binding method include methods similar to those listed as the method for immobilizing the first binding molecule on the carrier.

When the first binding molecule and/or the second binding molecule have a label, the kit of the present disclosure may have a substrate capable of reacting with the label. In this case, the label is, for example, the enzyme label described above, and the substrate is a substance capable of reacting with the enzyme label. The substrate may be in the form of a solid or a liquid. When the substrate is in the form of a liquid, the substrate can also be referred to as a “substrate liquid”.

The first binding molecule and/or the second binding molecule included in the kit of the present disclosure may be dissolved or dispersed in a buffer solution or the like and be provided in the form of a solution (a liquid or gel), or may be dissolved in a buffer solution or the like and be provided in the form of a solid such as powder or granules obtained by lyophilizing the resulting liquid. Examples of the buffer solution include Tris buffer solutions such as a Tris-HCl buffer solution, a Tris-EDTA (TE) buffer solution, a TAE buffer solution, a TBE buffer solution, and a Tris-buffered physiological saline solution; phosphate buffer solutions such as a phosphate-buffered physiological saline solution; carbonate buffer solutions such as a sodium carbonate-bicarbonate buffer solution; GOOD buffer solutions such as MES, ADA, PIPES, TAPS, CAPS, ACES, cholamine chloride, BES, TES, HEPES, acetamidoglycine, tricine, glycinamide, and bicine; and the like. The pH of the solution is, for example, 4.0 to 9.5, preferably 5 to 9 or 5.5 to 8.5, and more preferably 6 to 8. The pH value can be adjusted by, for example, using a buffer solution as described above, an acidic substance such as hydrochloric acid and an alkaline substance such as sodium hydroxide. Furthermore, the solution may contain a water-soluble macromolecule such as bovine serum albumin (BSA), a chelating agent such as EDTA, a sugar such as sucrose, or an antiseptic such as sodium azide.

The kit of the present disclosure may further include a standard ApoE. One or two or more aqueous solutions containing ApoE at predetermined concentrations or ApoE powder (e.g., lyophilized product) is used as the standard ApoE. The standard ApoE is useful as, for example, a control. Using the standard ApoE also makes it possible to, for example, produce a standard curve depending on the ApoE concentrations to analyze the ApoE concentration in a sample in an ApoE measurement method, which will be described below.

The kit of the present disclosure may also include, for example, a diluent for diluting a sample (specimen diluent), a diluent for diluting the first binding molecule (first binding molecule diluent), a diluent for diluting the second binding molecule (second binding molecule diluent), a washing solution for washing the complex formed through the reaction between the sample and the ApoE measurement reagent, a pretreatment solution (treatment solution), and the like.

The diluents above contain, for example, the buffer solution above. The washing solution above contains, for example, the buffer solution above, a nonionic surfactant, and the like.

In the kit of the present disclosure, the surfactant having a steroid skeleton may be included in the state of being separate from the ApoE measurement reagent or in the state of being mixed with a portion or all of the ApoE measurement reagent. It is preferable that the surfactant having a steroid skeleton coexists at the time of contact between the sample and the ApoE measurement reagent because the inhibition of ApoE measurement by a lipid can be suppressed. Accordingly, it is preferable that, at the time of contact between the sample and the ApoE measurement reagent or prior to the contact, the surfactant having a steroid skeleton is contained in a reagent used. Therefore, in the kit of the present disclosure, the surfactant having a steroid skeleton can be contained in, for example, the solution in which the first binding molecule is dissolved or dispersed, the specimen diluent, the first binding molecule diluent, the solution in which the second binding molecule is dissolved or dispersed, and/or the second binding molecule diluent according to the order in which the kit of the present disclosure is used. Specifically, when the kit of the present disclosure includes, as the ApoE measurement reagent, the first binding molecule, the specimen diluent, and optionally the second binding molecule, the sample is diluted with the specimen diluent, and then the obtained diluted solution is brought into contact with the first binding molecule, it is preferable that the surfactant is contained in the solution in which the first binding molecule is dissolved or dispersed, the specimen diluent, and/or the first binding molecule diluent. Accordingly, the kit of the present disclosure makes it possible to, for example, effectively suppress the inhibitory effect of a lipid on the measurement because the sample and the surfactant having a steroid skeleton can coexist.

In the kit of the present disclosure, the reagents or constituent components may be in the form of a solid such as powder or granules, or in the form of a liquid such as a slurry (suspension), a jelly, or a solution.

The constituent elements of the kit of the present disclosure may be, for example, included in the state of being separate from each other or in the state in which some or all of them are mixed or not mixed. When all the reagents of the kit of the present disclosure are contained in one container in the state of being mixed or not mixed, the kit of the present disclosure can also be referred to as, for example, the “reagent for use to measure an ApoE”.

The kit of the present disclosure may further include, for example, a container for storing the constituent components of the kit. In this case, the constituent components of the kit of the present disclosure may be provided in separate containers (e.g., tubes, plates, or the like). The kit of the present disclosure may also be provided in the form of a device. In this case, some or all of the constituent components may be provided in the form of being contained in the device. When some of the constituent components are provided in the form of being contained in a device, the other constituent components of the kit may be provided, for example, in the form of not being contained in the device such as the form of being contained in different containers. In this case, the constituent components that are not contained in the device may be used through injection into the device at the time of ApoE measurement. Examples of the structure of the device include those of devices as follows: 1) a device that includes a first compartment for preparing a mixed liquid by mixing a sample and the specimen diluent, and a second compartment for detecting an ApoE by bringing the prepared mixed liquid into contact with the surfactant having a steroid skeleton and the ApoE measurement reagent; 2) a device that includes a compartment for detecting an ApoE by mixing a sample, the surfactant having a steroid skeleton, and the ApoE measurement reagent; 3) a device that includes a channel that enables mixing of a sample and the constituent components (e.g., a reaction solution, a diluent, and the like), and a compartment for detecting an ApoE; and the like.

The kit of the present disclosure may include, for example, an instruction manual or an operation manual.

The kit of the present disclosure can be favorably used as an inspection kit, test kit, or research kit for use to, for example, detect an ApoE in a sample that contains lipids. The kit of the present disclosure can be used to favorably conduct an ApoE measurement method of the present disclosure, which will be described below.

### Method for Suppressing Inhibition of ApoE Measurement by Lipid

Another aspect of the present disclosure discloses a method capable of suppressing an inhibitory effect of a lipid on ApoE measurement. The method for suppressing inhibition of apolipoprotein E (ApoE) measurement by a lipid according to the present disclosure (this method may also be referred to as the “suppression method” hereinafter) includes a coexistence step of allowing a target sample to coexist with a surfactant having a steroid skeleton. With the suppression method of the present disclosure, it is possible to suppress the inhibitory effect of a lipid on the ApoE measurement. Accordingly, with the suppression method of the present disclosure, it is possible to, for example, favorably measure an ApoE in a sample even when the sample contains the lipid.

The suppression method of the present disclosure can also be referred to as, for example, a method for pretreating a sample for use in ApoE measurement, a method for preparing (producing) a lipid-containing sample capable of undergoing ApoE measurement, or a method for preparing (producing) a lipid-containing sample with an improved efficiency of ApoE measurement.

The suppression method of the present disclosure includes a coexistence step of allowing a target sample to coexist with a surfactant having a steroid skeleton. The coexistence above can be achieved through contact between or mixing of the target sample and the surfactant having a steroid skeleton (these operations are collectively referred to as “contact” hereinafter). It is preferable to achieve the coexistence above in a liquid system (coexistence system or mixing system) that contains water, a physiological saline solution, a buffer solution as described above, or the like. The order of the contact is not particularly limited, and any order can be employed.

In the coexistence step, the concentration of the surfactant having a steroid skeleton in the liquid system is, for example, 0.001 to 10 (w/v) %, 0.005 to 5 (w/v) %, or 0.010 to 3 (w/v) %, and is preferably 0.01 to 2.5 (w/v) % and more preferably 0.015 to 2 (w/v) % because the inhibition of ApoE measurement by a lipid can be further suppressed. The concentration of the surfactant can also be referred to as, for example, the “concentration in the liquid system”.

When the surfactant having a steroid skeleton is CHAPS, the concentration of CHAPS in the liquid system is, for example, 0.001 to 10 (w/v) %, 0.005 to 5 (w/v) %, or 0.01 to 3 (w/v) %, is preferably 0.23 to 1.9 (w/v) % because the inhibition of ApoE measurement by a lipid can be further suppressed, and is more preferably 0.24 to 1.9 (w/v) % because the inhibition of ApoE4 measurement and total ApoE measurement by a lipid can be further suppressed.

When the surfactant having a steroid skeleton is deoxycholic acid, the concentration of deoxycholic acid in the liquid system is, for example, 0.001 to 10 (w/v) %, 0.005 to 5 (w/v) %, or 0.010 to 3 (w/v) %, is preferably 0.019 to 0.29 (w/v) % because the inhibition of ApoE measurement by a lipid can be further suppressed, and is more preferably 0.019 to 0.23 (w/v) % or 0.038 to 0.23 (w/v) % because the inhibition of ApoE4 measurement and total ApoE measurement by a lipid can be further suppressed.

When the surfactant having a steroid skeleton is cholic acid, the concentration of cholic acid in the liquid system is, for example, 0.001 to 10 (w/v) %, 0.005 to 5 (w/v) %, or 0.010 to 3 (w/v) %, is preferably 0.028 to 1.9 (w/v) % because the inhibition of ApoE measurement by a lipid can be further suppressed, and is more preferably 0.029 to 1.9 (w/v) % or 0.095 to 1.9 (w/v) % because the inhibition of ApoE4 measurement and total ApoE measurement by a lipid can be further suppressed.

When the surfactant having a steroid skeleton is glycocholic acid, the concentration of glycocholic acid in the liquid system is, for example, 0.001 to 10 (w/v) %, 0.005 to 5 (w/v) %, or 0.010 to 3 (w/v) %, is preferably 0.028 to 0.29 (w/v) % or 0.047 to 0.29 (w/v) % because the inhibition of ApoE measurement by a lipid can be further suppressed, and is more preferably 0.029 to 0.19 (w/v) % or 0.095 to 0.19 (w/v) % because the inhibition of ApoE4 measurement and total ApoE measurement by a lipid can be further suppressed.

When the surfactant having a steroid skeleton is taurocholic acid, the concentration of taurocholic acid in the liquid system is, for example, 0.001 to 10 (w/v) %, 0.005 to 5 (w/v) %, or 0.01 to 3 (w/v) %, is preferably 0.023 to 0.29 (w/v) % or 0.047 to 0.29 (w/v) % because the inhibition of ApoE measurement by a lipid can be further suppressed, and is more preferably 0.029 to 0.19 (w/v) % or 0.048 to 0.19 (w/v) % because the inhibition of ApoE4 measurement and total ApoE measurement by a lipid can be further suppressed.

In the coexistence step, the conditions (e.g., temperature, period, and pH) for the coexistence are not limited as long as the surfactant having a steroid skeleton can suppress inhibition of ApoE measurement by a lipid. Specifically, the temperature during the coexistence is, for example, 4 to 42° C. or 18 to 40° C. The period of the coexistence is, for example, 1 minute to 12 hours, 3 minutes to 120 minutes, or 5 to 60 minutes. The pH during the coexistence is, for example, 4 to 9.5, preferably 5 to 9 or 5.5 to 8.5, and more preferably 6 to 8.

The surfactant having a steroid skeleton may be contained in, for example, the diluent. In this case, in the coexistence step, the sample and the surfactant having a steroid skeleton are allowed to coexist by, for example, diluting the sample with the diluent. In the dilution above, the volume of the diluent used to dilute the sample can be determined as appropriate depending on the volume of the sample and the intended use (e.g., qualitative measurement, quantitative measurement, or the like) of the sample, and can be set to, for example, a volume larger than the volume of the sample. Specifically, the volume of the diluent used to dilute the sample is, for example, 1 to 25 times, preferably 2 to 20 times, more preferably 4 to 10 times, as large as the volume of the sample, which is defined as a standard (1).

The suppression of inhibition of ApoE measurement can also be considered as promotion of a reaction between the ApoE and the reagent. The suppression of inhibition of ApoE measurement can be evaluated by, for example, using a lipid-rich blood sample to which chyle has been added and a control sample to which the chyle has not been added in accordance with Example 1, which will be described below. Specifically, in the evaluation above, for example, first, a control blood sample to which a test substance has been added such that the concentration in the liquid system is 0.2 (w/v) % or the test substance has not been added and a lipid-rich blood sample to which the test substance has been added such that the concentration in the liquid system is 0.2 (w/v) % or the test substance has not been added are prepared, and the ApoE measurement values (concentrations) are acquired from the samples. Next, a ratio (C) of the measurement value of the lipid-rich blood sample to which the test substance has not been added to the ApoE measurement value (100%) of the control blood sample to which the test substance has not been added and a ratio (T) of the measurement value of the lipid-rich blood sample to which the test substance has been added to the ApoE measurement value (100%) of the control blood sample to which the test substance has been added are calculated. Then, when the ratio T is greater than the ratio C (for example, when the difference between the ratio T and the ratio C (T-C) is 2.5% or more, 5% or more, 10% or more, 15% or more, 20% or more, or 25% or more), it can be determined that the test substance can suppress the inhibition of ApoE measurement.

When suppression of the inhibition of ApoE measurement is evaluated in a measurement method, which will be described below, the evaluation may be conducted in the presence of a measurement reagent, which will be described below. In this case, in the evaluation above, for example, the ApoE measurement values (concentrations) may be acquired from a control blood sample under the condition in which a test substance has been added such that the concentration in a reaction solution in the measurement method, which will be described below, is 0.2 (w/v) % or the test substance has not been added, and a lipid-rich blood sample under the condition in which a test substance has been added such that the concentration in the reaction solution is 0.2 (w/v) % or the test substance has not been added. Next, a ratio (C) of the measurement value of the lipid-rich blood sample under the condition in which the test substance has not been added to the ApoE measurement value (100%) of the control blood sample under the condition in which the test substance has not been added and a ratio (T) of the measurement value of the lipid-rich blood sample under the condition in which the test substance has been added to the ApoE measurement value (100%) of the control blood sample under the condition in which the test substance has been added may be calculated. Then, the obtained ratio (C) and ratio (T) can be evaluated based on, for example, the evaluation criteria described above.

### Method for Measuring ApoE

Another aspect of the present disclosure provides a method for measuring an ApoE while suppressing an inhibitory effect of a lipid on the ApoE measurement. The method for measuring an ApoE according to the present disclosure (this method may also be referred to as the “measurement method” hereinafter) includes a measurement step of measuring an ApoE in a target sample by bringing the sample into contact with an ApoE measurement reagent in the presence of a surfactant having a steroid skeleton. With the measurement method of the present disclosure, it is possible to suppress the inhibitory effect of a lipid on the ApoE measurement because the ApoE is measured by bringing the ApoE into contact with the ApoE measurement reagent in the presence of the surfactant having a steroid skeleton. Accordingly, with the measurement method of the present disclosure, it is possible to, for example, favorably measure an ApoE in a sample even when the sample contains the lipid.

In the measurement step, the ApoE in the sample is measured by bringing the sample into contact with the ApoE measurement reagent in the presence of the surfactant having a steroid skeleton. In the measurement step, it is possible to, for example, qualitatively measure the presence of an ApoE in the sample by, for example, measuring the presence of an ApoE detected using the ApoE measurement reagent. In the measurement step, it is possible to quantitatively measure the amount of an ApoE in the sample by, for example, measuring the amount of an ApoE detected using the ApoE measurement reagent. In the measurement step, the measurement target ApoE may be any one type of ApoE, a plurality of types of ApoEs, or all types of ApoEs (total ApoE) as described above.

In the measurement step, the ApoE measurement can be conducted using a binding molecule for the ApoE. In this case, the ApoE measurement may be conducted using an immunological technique. Examples of the immunological technique include direct competitive ELISA, indirect competitive ELISA, sandwich ELISA, direct competitive immunoassay, indirect competitive immunoassay, sandwich immunoassay, immunochromatography, spin immunoassay, and latex agglutination. When the ApoE measurement reagent is a reagent with a label, the immunological technique is fluorescent immunoassay (FIA), enzyme immunoassay (EIA), chemiluminescent immunoassay, chemiluminescent enzyme immunoassay, or radioimmunoassay (RIA) depending on the type of label.

The following describes, as examples of the measurement step, a measurement example in which the ApoE is measured using the first binding molecule, and a measurement example in which the ApoE is measured using the first binding molecule and the second binding molecule.

When the ApoE is measured using the first binding molecule, the measurement step includes, for example, a first complex formation step of forming a first complex composed of an ApoE in a sample and a first binding molecule for an ApoE by bringing the sample into contact with the first binding molecule in the presence of a surfactant having a steroid skeleton, and a complex measurement step of measuring the ApoE in the sample by measuring the first complex.

In the complex formation step, the contact above can be achieved by, for example, mixing the target sample and the first binding molecule for an ApoE. It is preferable to achieve the contact above in a liquid system that contains water, a physiological saline solution, a buffer solution as described above, or the like.

In the complex formation step, the concentration of the surfactant having a steroid skeleton in the liquid system is, for example, 0.001 to 10 (w/v) %, 0.005 to 5 (w/v) %, or 0.010 to 3 (w/v) %, and is preferably 0.01 to 2.5 (w/v) % and more preferably 0.015 to 2 (w/v) % because the inhibition of ApoE measurement by a lipid can be further suppressed. The concentration of the surfactant can also be referred to as, for example, “the concentration in the liquid system (reaction solution) containing the sample and the first binding molecule for an ApoE”.

When the surfactant having a steroid skeleton is CHAPS, the concentration of CHAPS in the liquid system is, for example, 0.001 to 10 (w/v) %, 0.005 to 5 (w/v) %, or 0.01 to 3 (w/v) %, is preferably 0.23 to 1.9 (w/v) % because the inhibition of ApoE measurement by a lipid can be further suppressed, and is more preferably 0.24 to 1.9 (w/v) % because the inhibition of ApoE4 measurement and total ApoE measurement by a lipid can be further suppressed.

When the surfactant having a steroid skeleton is deoxycholic acid, the concentration of deoxycholic acid in the liquid system is, for example, 0.001 to 10 (w/v) %, 0.005 to 5 (w/v) %, or 0.010 to 3 (w/v) %, is preferably 0.019 to 0.29 (w/v) % because the inhibition of ApoE measurement by a lipid can be further suppressed, and is more preferably 0.019 to 0.23 (w/v) % or 0.038 to 0.23 (w/v) % because the inhibition of ApoE4 measurement and total ApoE measurement by a lipid can be further suppressed.

When the surfactant having a steroid skeleton is cholic acid, the concentration of cholic acid in the liquid system is, for example, 0.001 to 10 (w/v) %, 0.005 to 5 (w/v) %, or 0.010 to 3 (w/v) %, is preferably 0.028 to 1.9 (w/v) % because the inhibition of ApoE measurement by a lipid can be further suppressed, and is more preferably 0.029 to 1.9 (w/v) % or 0.095 to 1.9 (w/v) % because the inhibition of ApoE4 measurement and total ApoE measurement by a lipid can be further suppressed.

When the surfactant having a steroid skeleton is glycocholic acid, the concentration of glycocholic acid in the liquid system is, for example, 0.001 to 10 (w/v) %, 0.005 to 5 (w/v) %, or 0.010 to 3 (w/v) %, is preferably 0.028 to 0.29 (w/v) % or 0.047 to 0.29 (w/v) % because the inhibition of ApoE measurement by a lipid can be further suppressed, and is more preferably 0.029 to 0.19 (w/v) % or 0.095 to 0.19 (w/v) % because the inhibition of ApoE4 measurement and total ApoE measurement by a lipid can be further suppressed.

When the surfactant having a steroid skeleton is taurocholic acid, the concentration of taurocholic acid in the liquid system is, for example, 0.001 to 10 (w/v) %, 0.005 to 5 (w/v) %, or 0.01 to 3 (w/v) %, is preferably 0.023 to 0.29 (w/v) % or 0.047 to 0.29 (w/v) % because the inhibition of ApoE measurement by a lipid can be further suppressed, and is more preferably 0.029 to 0.19 (w/v) % or 0.048 to 0.19 (w/v) % because the inhibition of ApoE4 measurement and total ApoE measurement by a lipid can be further suppressed.

In the complex formation step, the conditions (e.g., temperature, period, and pH) for the contact between the sample and the first binding molecule are not limited as long as the surfactant having a steroid skeleton can suppress inhibition of ApoE measurement by a lipid and the first complex can be formed. Specifically, the temperature during the contact is, for example, 4 to 42° C. or 18 to 40° C. The period of the contact is, for example, 1 minute to 12 hours, 3 minutes to 120 minutes, or 5 to 60 minutes. The pH during the formation of the complex is, for example, 4 to 9.5, preferably 5 to 9 or 5.5 to 8.5, and more preferably 6 to 8.

In the measurement step or the complex formation step, it is sufficient that the surfactant having a steroid skeleton is present at the time of the contact between the sample and the ApoE measurement reagent (e.g., the first binding molecule and/or the second binding molecule), and the order of the contact is not particularly limited. In the measurement step or the complex formation step, for example, the following procedure may be conducted: the surfactant having a steroid skeleton and the sample are first brought into contact with each other and then further brought into contact with the ApoE measurement reagent (e.g., a solution containing the first binding molecule and/or the second binding molecule), or the surfactant having a steroid skeleton and the ApoE measurement reagent (e.g., a solution containing the first binding molecule and/or the second binding molecule) are first brought into contact with each other and then brought into contact with the sample, or the surfactant having a steroid skeleton, the sample, and the ApoE measurement reagent (e.g., a solution containing the first binding molecule and/or the second binding molecule) are brought into contact with one another at the same time. Also, in the measurement step or the complex formation step, the following procedure may also be conducted: the sample and the surfactant having a steroid skeleton are allowed to coexist by diluting the sample with a specimen diluent containing the surfactant having a steroid skeleton, and then the resulting mixture is brought into contact with the ApoE measurement reagent (e.g., a solution containing the first binding molecule and/or the second binding molecule).

In the dilution above, the volume of the diluent used to dilute the sample can be determined as appropriate depending on the volume of the sample and the intended use (e.g., qualitative measurement, quantitative measurement, or the like) of the sample, and can be set to, for example, a volume larger than the volume of the sample. Specifically, the volume of the diluent used to dilute the sample is, for example, 1 to 25 times, preferably 2 to 20 times, more preferably 4 to 10 times, as large as the volume of the sample, which is defined as a standard (1).

The complex measurement step is, for example, a step of measuring the first complex composed of an ApoE in the sample and the first binding molecule, that is, a step of measuring binding between the ApoE and the first binding molecule. In the complex measurement step, detecting the presence of the binding between the ApoE and the first binding molecule makes it possible to, for example, analyze the presence of an ApoE in the sample (qualitative analysis), and detecting the degree (level) of the binding between the ApoE and the first binding molecule makes it possible to, for example, analyze the amount of an ApoE in the sample (quantitative analysis).

There is no particular limitation on the method for measuring binding between the ApoE and the first binding molecule. For example, a conventionally known method for measuring binding between substances can be employed, and specific examples thereof include SPR and fluorescence polarization assay. When the first binding molecule has the label, binding between the ApoE and the first binding molecule may be measured in the complex measurement step by directly or indirectly detecting the label of the first complex. The detection of the label can be determined as appropriate, for example, depending on the type of label.

When the ApoE is measured using the first binding molecule and the second binding molecule, the measurement step includes a first complex formation step of forming a first complex composed of an ApoE in the sample and a first binding molecule for an ApoE by bringing the sample into contact with the first binding molecule in the presence of the surfactant having a steroid skeleton, a second complex formation step of forming a second complex composed of the first complex and a second binding molecule for an ApoE by bringing the first complex into contact with the second binding molecule, and a complex measurement step of measuring the ApoE in the sample by measuring the second complex. In this case, it is preferable that the first binding molecule is supported by the carrier before, during, or after the formation of the first complex. When the first binding molecule is supported by the carrier after the formation of the first complex, the first binding molecule is, for example, supported by the carrier in the second complex formation step, which will be described below, in the measurement method. When the first binding molecule is supported by the carrier during or after the formation of the first complex, the first binding molecule is, for example, indirectly immobilized on the carrier. The indirect immobilization above can be achieved by, for example, adding the pair of affinity substances described above to the first binding molecule and the carrier.

The first complex formation step can be conducted similarly to the complex formation step above. In the measurement step, it is preferable to separate the first complex after the first complex formation step because the accuracy of the ApoE measurement can be improved, and it is more preferable to optionally further wash the separated first complex with the washing solution. The first complex can be separated through, for example, solid-liquid separation. When the first binding molecule is supported by the carrier, the first complex can be separated by, for example, separating a solid fraction containing the carrier from a liquid fraction. When the first binding molecule is supported by a magnetic particle, the first complex can be separated by generating a magnetic field using a magnet or the like and thus separating a solid fraction containing the magnetic particle from a liquid fraction.

Next, in the second complex formation step, the second complex is formed by bringing the first complex into contact with the second binding molecule. It is preferable to achieve the contact between the first complex and the second binding molecule, for example, in a liquid system that contains water, a physiological saline solution, a buffer solution as described above, or the like.

In the second complex formation step, the conditions for the contact between the first complex and the second binding molecule are not particularly limited as long as the second complex can be formed. Specifically, the temperature during the contact is, for example, 4 to 42° C. or 18 to 40°° C. The period of the contact is, for example, 1 minute to 12 hours, 3 minutes to 120 minutes, or 5 minutes to 60 minutes.

In the measurement step, it is preferable to separate the second complex after the second complex formation step because the accuracy of the ApoE measurement can be improved, and it is more preferable to optionally further wash the separated second complex with the washing solution. The separation of the second complex can be conducted, for example, similarly to the separation of the first complex.

The complex measurement step is, for example, a step of measuring the second complex composed of the first complex and the second binding molecule, that is, a step of measuring binding between the first complex and the second binding molecule. In the complex measurement step, detecting the presence of the binding between the first complex and the second binding molecule makes it possible to, for example, analyze the presence of an ApoE in the sample (qualitative analysis), and detecting the degree (level) of the binding between the first complex and the second binding molecule makes it possible to, for example, analyze the amount of an ApoE in the sample (quantitative analysis). The method for measuring binding between the first complex and the second binding molecule can be conducted, for example, similarly to the method for measuring binding between the ApoE and the first binding molecule. When the second binding molecule has the label, binding between the first complex and the second binding molecule may be measured in the complex measurement step by directly or indirectly detecting the label of the second complex. The detection of the label can be determined as appropriate, for example, depending on the type of label.

In the complex measurement step, the binding between the first complex and the second binding molecule may be measured by, for example, separating the second complex composed of the first complex and the second binding molecule, washing the separated second complex with the washing solution, causing the label to be released from the second complex, and detecting the released label.

In the measurement method of the present disclosure, the first complex formation step and the second complex formation step may be conducted at the same time. When the first and second complex formation steps are conducted at the same time, these steps can be conducted similarly to the complex formation step above.

As described above, with the measurement method of the present disclosure, it is possible to measure the ApoE.

In the measurement method of the present disclosure, for example, the amount of an ApoE may be optionally analyzed in the measurement step. In this case, in the measurement step, for example, the correlation between the abundance of the ApoE and the measurement value obtained through ApoE measurement is determined in advance, and then the amount of an ApoE in the sample can be analyzed using the measurement value based on the correlation. Accordingly, the measurement method of the present disclosure can also be referred to as, for example, the “analysis method”.

In the measurement method of the present disclosure, for example, the genotype of the ApoE may be optionally analyzed in the measurement step. The genotype of the ApoE can be analyzed, for example, in accordance with the analysis method described in Non-Patent Literature 2 above. Specifically, the amount of the total ApoE and the amount of the ApoE (e.g., ApoE2, ApoE3, ApoE4, or the like) of the analysis target genotype are measured using the measurement method of the present disclosure. With the measurement method of the present disclosure, it is possible to determine if the ApoE is homozygous or heterozygous for the analysis target genotype or does not have this genotype based on the ratio of the ApoEs of the analysis target genotype in the total ApoE.

With the measurement method of the present disclosure, it is possible to, for example, favorably measure the ApoE even in a sample containing the lipid, such as a biological sample (e.g., a blood sample). Accordingly, the measurement method of the present disclosure can be favorably used as, for example, a method for detecting or inspecting an ApoE in the biological sample.

### Test Method

Another aspect of the present disclosure provides a method for evaluating a risk, or testing a likelihood, that amyloid-related imaging abnormalities occur when a disease-modifying drug for Alzheimer's disease such as anti-amyloid β antibody is administered to a target. The test method of the present disclosure is a method for testing a likelihood that amyloid-related imaging abnormalities occur when a disease-modifying drug for Alzheimer's disease is administered to a target, and includes a measurement step of measuring an ApoE in a sample from the target by bringing the sample into contact with an ApoE measurement reagent in the presence of a surfactant having a steroid skeleton. With the test method of the present disclosure, it is possible to evaluate a risk that ARIA occurs, or test a likelihood that ARIA occurs, in the target (administration target) when a disease-modifying drug for AD is administered.

The description of the measurement step of the measurement method of the present disclosure can be applied to the measurement step of the test method of the present disclosure.

Examples of the disease-modifying drug for AD include anti-amyloid β antibodies, and specific examples thereof include lecanemab (Leqembi (registered trademark), donanemab, and aducanumab.

The test method of the present disclosure may include, for example, an evaluation step of evaluating a likelihood that ARIA occurs based on the analysis results of the ApoE genotype obtained in the measurement step. Specifically, when, for example, the genotype of the ApoE of the target is heterozygous for the ApoE4 or homozygous for the ApoE4, it is possible to determine in the evaluation step that the target is more likely to develop ARIA when the disease-modifying drug for AD is administered. On the other hand, when, for example, the genotype of the ApoE of the target does not have the ApoE4, it is possible to determine that the target is less likely to develop ARIA when the disease-modifying drug for AD is administered.

The test method of the present disclosure may include, for example, an administration step of administering the disease-modifying drug for AD based on evaluation results obtained in the evaluation step. Specifically, in the administration step, the disease-modifying drug for AD is administered to, for example, a target that is less likely to develop ARIA. When the test method of the present disclosure includes the administration step, the test method of the present disclosure can also be referred to as, for example, “a companion diagnostic method for Alzheimer's disease”, “a method for testing and treating Alzheimer's disease”, or “a method for diagnosing and treating Alzheimer's disease”.

The test method of the present disclosure may include, for example, based on the results obtained in the evaluation step, determining whether or not the disease-modifying drug for AD is to be administered, determining whether or not the dosage of the disease-modifying drug for AD is to be increased or reduced relative to the reference value of the dosage of the disease-modifying drug for AD, and/or determining a follow-up protocol for the target to which the disease-modifying drug for AD has been administered.

### Use

An aspect of the present disclosure is use of the composition of the present disclosure for use to suppress inhibition of apolipoprotein E (ApoE) measurement by a lipid. Another aspect of the present disclosure is use of the kit of the present disclosure for use to measure an ApoE while suppressing an inhibitory effect of a lipid on ApoE measurement.

### EXAMPLES

Hereinafter, the present disclosure will be described in detail by way of examples, but the present disclosure is not limited to aspects described in the examples. Commercially available reagents and kits were used in accordance with the attached protocols unless otherwise stated. In the following descriptions, “mol/l” may also be referred to as “M”.

### Example 1

It was confirmed using blood samples that the ApoE4 measurement values and the Pan-ApoE measurement values decreased in the presence of a lipid, that is, the measurement was inhibited, and addition of CHAPS made it possible to suppress the inhibition of the measurement by a lipid.

For each of blood samples, which will be described below, preparation of an immobilized particle solution, ApoE4 measurement, and Pan-ApoE measurement were conducted in accordance with the following procedures.

### (1) Preparation of Immobilized Particle Solution

Magnetic particles on which a mouse anti-ApoE4 antibody, which specifically binds to the ApoE4, had been immobilized were suspended in a particle diluent (50 mM Tris buffer solution, 1 mM EDTA 2Na, 0.1% NaN₃, 2.0% BSA, pH 7.2), and thus an anti-ApoE4 antibody-immobilized particle solution was obtained. Note that a reagent contained in Lumipulse (registered trademark) G ApoE4 Immunoreaction Cartridges (manufactured by Fujirebio Europe N.V.) was used as the magnetic particles to which a mouse anti-ApoE4 antibody had been immobilized.

An anti-Pan-ApoE antibody-immobilized particle solution was prepared in the same manner using a mouse anti-Pan-ApoE antibody. The anti-Pan-ApoE antibody is an antibody that binds to not only the ApoE4 but also ApoE2 and ApoE3, which are derived from other alleles. Note that a reagent contained in Lumipulse (registered trademark) G Pan-ApoE Immunoreaction Cartridges (manufactured by Fujirebio Europe N.V.) was used as the magnetic particles to which a mouse anti-Pan-ApoE antibody had been immobilized.

### (2) ApoE4 Measurement Method

20 μL of a sample was diluted with 180 μL of a specimen diluent. 10 μL of the obtained diluted solution of the sample was removed, and was dispensed into a reaction vessel in which 150 μL of the anti-ApoE4 antibody-immobilized particle solution had been placed. The obtained reaction solution (solution B) was stirred and was then incubated at 37° C. for 10 minutes. After the incubation, bead fraction/fluid fraction (B/F) separation was conducted using a magnetic field, and the bead fraction was collected and washed with a washing solution. Next, 150 μL of an enzyme-labeled antibody solution containing an alkaline phosphatase (ALP)-labeled anti-ApoE monoclonal antibody was dispensed into the reaction vessel, and the resultant mixture was stirred and was then incubated at 37° C. for 10 minutes. After the incubation, as described above, the B/F separation was conducted using a magnetic field, and then the washing was conducted. Next, 200 μL of a Lumipulse (registered trademark) substrate solution (manufactured by Fujirebio Inc.) containing a chemiluminescent substrate, 3-(2′-spiroadamantane)-4-methoxy-4-(3″-phosphoryloxy) phenyl-1,2-dioxetane disodium salt (AMPPD), was dispensed into the reaction vessel, and the resultant mixture was stirred and was then incubated at 37° C. for 5 minutes. After the incubation, the luminescence level of the resultant reaction solution was measured using a luminometer. The measurement above was conducted using a fully automated chemiluminescence enzyme immunoassay system (Lumipulse G1200 (manufactured by Fujirebio Inc.)). In addition, a reagent contained in Lumipulse G ApoE4 Immunoreaction Cartridges was used as the enzyme-labeled antibody solution. As the specimen diluent, the washing solution, and the substrate solution, the Lumipulse reagents (manufactured by Fujirebio Inc.) were used in accordance with the manual attached to Lumipulse G ApoE4 Immunoreaction Cartridges.

The measurement was conducted in accordance with the protocol attached to Lumipulse G ApoE4 Calibrators (Fujirebio Europe N.V.) and a calibration curve was produced. The ApoE4 measurement values of the blood samples were calculated from the count values of the blood samples based on the calibration curve.

### (3) Pan-ApoE Measurement Method

20 μL of a sample was diluted with 180 μL of a specimen diluent. 10 μL of the obtained diluted solution of the sample was removed, and was dispensed into a reaction vessel in which 200 μL of the anti-Pan-ApoE antibody-immobilized particle solution had been placed. The obtained reaction solution (solution C) was stirred and was then incubated at 37° C. for 10 minutes. After the incubation, as described above, the B/F separation was conducted using a magnetic field, and then the washing was conducted. Next, 150 μL of an enzyme-labeled antibody solution containing an ALP-labeled anti-ApoE monoclonal antibody was dispensed into the reaction vessel, and the resultant mixture was stirred and was then incubated at 37° C. for 10 minutes. After the incubation, as described above, the B/F separation was conducted using a magnetic field, and then the washing was conducted. Next, 200 μL of a Lumipulse (registered trademark) substrate solution (manufactured by Fujirebio Inc.) containing a chemiluminescent substrate, AMPPD, was dispensed into the reaction vessel, and the resultant mixture was stirred and was then incubated at 37° C. for 5 minutes. After the incubation, the luminescence level of the resultant reaction solution was measured using a luminometer. The measurement above was conducted using a fully automated chemiluminescence enzyme immunoassay system. A reagent contained in Lumipulse G Pan-ApoE Immunoreaction Cartridges was used as the enzyme-labeled antibody solution. As the specimen diluent, the washing solution, and the substrate solution, the Lumipulse reagents (manufactured by Fujirebio Inc.) were used in accordance with the manual attached to Lumipulse G Pan-ApoE Immunoreaction Cartridges.

The measurement was conducted in accordance with the protocol attached to Lumipulse G Pan-ApoE Calibrators (Fujirebio Europe N.V.) and a calibration curve was produced. The Pan-ApoE measurement values of the blood samples were calculated from the count values of the blood samples based on the calibration curve.

### (4) Examination of Inhibition of Measurement by Lipid, and Examination of Additive Capable of Suppressing Inhibition of Measurement

The inventors of the present invention found that some blood samples measured using the measurement method above decreased in the ApoE4 measurement values and the Pan-ApoE measurement values. As a result of examination of the reasons therefor, the inventors of the present invention came up with an idea that a blood sample containing lipids such as a neutral fat at high concentrations decreases in the measurement value. Therefore, in this example, it was examined whether the ApoE4 measurement values and the Pan-ApoE measurement values decreased due to addition of a lipid, and which additive was capable of inhibiting the decrease in measurement values.

Blood samples (lipid-rich samples) containing lipids at a high concentration were prepared by adding 20 μl of chyle contained in Interference Check A Plus (manufactured by Sysmex Corporation) to 180 μl of blood samples (EDTA 2K plasma) 1 to 3 containing ApoE4 and Pan-ApoE. A control blood sample was prepared by adding the blank sample contained in Interference Check A Plus above in the same manner. The obtained blood samples and a specimen diluent were used in accordance with the above-described preparation of immobilized particle solution, ApoE4 measurement, and Pan-ApoE measurement to acquire ApoE4 measurement values and Pan-ApoE measurement values. A surfactant such as Tween20, Tergitol 15-S-30, CHAPS, hexadecyltrimethylammonium chloride (C16TAC), or SDS (sodium dodecyl sulfate) was used as the additive, and was added to the particle diluent such that the concentration in the antibody-immobilized particle solution (solution A) was 1.0 w/v %. For a control of the additive, measurement was conducted in the same manner, except that the antibody-immobilized particle solution to which the surfactant had not been added was used. Then, in order to detect the presence of inhibition of measurement, a ratio of a measurement value (L) of the lipid-rich sample to a measurement value (C) of the control (L/C×100 (%)) was calculated from the obtained measurement values. In addition, the coefficient of variation (CV) was calculated from L/C of each sample to evaluate the variation of the effect of suppressing inhibition of the measurement by a lipid. Table 1 below shows the ApoE4 measurement values, and Table 2 below shows the Pan-ApoE measurement values.

TABLE 1

| | | Not | | | | | |
|---|---|---|---|---|---|---|---|
| ApoE4 | | added | Non-Ionic | | Amphoteric | Cationic | Anionic |
| Surfactant concentration | | — | Tween20 | Tergitol | CHAPS | C16TAC | SDS |
| Solution A (1.0 w/v %) | | | | 15-S-30 | | | |
| Surfactant concentration | | | | | | | |
| Solution B (0.94 w/v %) | | | | | | | |
| Comparison of | Average | 67% | 92% | 84% | 97% | 90% | 793% |
| measurement | value | | | | | | |
| values (%) | CV | 9% | 6% | 3% | 3% | 2% | 648% |
| Lipid-rich/ | | | | | | | |
| control sample | | | | | | | |

TABLE 2

| | | Not | | | | | |
|---|---|---|---|---|---|---|---|
| Pan-ApoE | | added | Non-Ionic | | Amphoteric | Cationic | Anionic |
| Surfactant concentration | | — | Tween20 | Tergitol | CHAPS | C16TAC | SDS |
| Solution A (1.0 w/v %) | | | | 15-S-30 | | | |
| Surfactant concentration | | | | | | | |
| Solution C (0.95 w/v %) | | | | | | | |
| Comparison of | Average | 60% | 94% | 78% | 97% | 79% | 363% |
| measurement | value | | | | | | |
| values (%) | CV | 14% | 21% | 16% | 4% | 32% | 311% |
| Lipid-rich/ | | | | | | | |
| control sample | | | | | | | |

As shown in Table 1 above, the measurement was inhibited by a lipid under the condition in which no surfactant had been added, and the ApoE4 measurement value decreased by about 22 to 45%. In contrast, the impact of the inhibition of the measurement by a lipid was suppressed under the condition in which CHAPS had been added to the specimen diluent, and the ApoE4 measurement value increased and recovered to the same level (within ±6%) as the measurement value before the addition of the lipid (control sample). On the other hand, the measurement value decreased under the condition in which another nonionic surfactant (Tween20, Tergitol 15-S-30) or cationic surfactant (C16TAC) had been added to the specimen diluent, and it was found that the degree of suppression of the inhibition of the measurement by a lipid is lower compared with CHAPS. In addition, the CV was significantly large and the measurement value greatly varied under the condition in which the anionic surfactant was added to the specimen diluent, and it was found that it is difficult to use the anionic surfactant in the ApoE4 measurement system.

As shown in Table 2 above, in the case of the Pan-ApoE as well, the measurement was inhibited by a lipid under the condition in which no surfactant had been added, and the Pan-ApoE measurement value decreased by 22 to 56%. In contrast, the impact of the inhibition of the measurement by a lipid was suppressed under the condition in which CHAPS had been added to the specimen diluent, and the measurement value increased and recovered to the same level (within ±8%) as the measurement value before the addition of the lipid (control sample). On the other hand, the recovery of the measurement value was 34% to 123% and greatly varied between the samples under the condition in which another nonionic surfactant (Tween20, Tergitol15-S-30) or cationic surfactant (C16TAC) had been added to the specimen diluent, and it was found that these surfactants cannot stably suppress the inhibition of the measurement by a lipid. In addition, the CV was significantly large and the measurement value greatly varied under the condition in which the anionic surfactant was added to the specimen diluent, and it was found that it is difficult to use the anionic surfactant in the Pan-ApoE measurement system.

It was found that the ApoE4 measurement and the Pan-ApoE measurement are inhibited in the presence of a lipid such as a neutral lipid, and addition of CHAPS makes it possible to suppress the inhibition of the measurement by a lipid.

### Example 2

It was confirmed that adding a surfactant having a steroid skeleton to a blood sample made it possible to suppress inhibition of ApoE4 measurement and Pan-ApoE measurement by a lipid.

CHAPS is a surfactant having a steroid skeleton, and examples of a surfactant having a similar skeleton include bile acid-based surfactants such as cholic acid, deoxycholic acid, glycocholic acid, and taurocholic acid. Accordingly, it was examined whether the ApoE4 measurement method described in Example 1 (2) above and the Pan-ApoE measurement method described in Example 1 (3) above could be used to suppress inhibition of measurement by a lipid in the same manner as in Example 1 above, except that the bile acid-based surfactants were used as the surfactants. Blood samples were prepared in the same manner as the method described in Example 1 above. Tables 3 to 7 below show the ApoE4 measurement results, and Tables 8 to 12 below show the Pan-ApoE measurement results.

TABLE 3

| | | Not | | | | |
|---|---|---|---|---|---|---|
| ApoE4 | | added | Deoxycholic acid | | | |
| Surfactant concentration | | — | 0.020% | 0.030% | 0.040% | 0.050% |
| Solution A (w/v %) | | | | | | |
| Surfactant concentration | | — | 0.019% | 0.028% | 0.038% | 0.047% |
| Solution B (w/v %) | | | | | | |
| Comparison of | Average | 77% | 92% | 96% | 101% | 114% |
| measurement | value | | | | | |
| values (%) | CV | 6% | 1% | 0% | 3% | 1% |
| Lipid-rich/ | | | | | | |
| control sample | | | | | | |

TABLE 4

| | | |
|---|---|---|
| | Not | |
| ApoE4 | added | Deoxycholic acid |

| Surfactant concentration | | | — | 0.10% | 0.25% |
|---|---|---|---|---|---|
| Solution A (w/v %) | | | | | |
| Surfactant concentration | | | — | 0.094% | 0.23% |
| Solution B (w/v %) | | | | | |
| | Comparison of | Average | 59% | 103% | 106% |
| | measurement | value | | | |
| | values (%) | CV | 8% | 10% | 11% |
| | Lipid-rich/ | | | | |
| | control sample | | | | |

As shown in Tables 3 and 4 above, it was found that, under all the concentration conditions (antibody-immobilized particle solution: 0.020 to 0.25%, reaction solution: 0.019 to 0.23%), deoxycholic acid could suppress the measurement interference in the ApoE4 measurement by a lipid compared with the condition in which no surfactant had been added. Also, it was found that, when deoxycholic acid was used, CV was sufficiently low under all the concentration conditions, and the inhibition of the ApoE measurement could be suppressed irrespective of the samples.

TABLE 5

| | | Not | | | | |
|---|---|---|---|---|---|---|
| ApoE4 | | added | Cholic acid | | | |
| Surfactant concentration | | — | 0.030% | 0.050% | 0.10% | 0.20% |
| Solution A (w/v %) | | | | | | |
| Surfactant concentration | | — | 0.028% | 0.047% | 0.094% | 0.19% |
| Solution B (w/v %) | | | | | | |
| Comparison of | Average | 70% | 80% | 91% | 108% | 108% |
| measurement | value | | | | | |
| values (%) | CV | 12% | 9% | 2% | 8% | 3% |
| Lipid-rich/ | | | | | | |
| control sample | | | | | | |
| | | Not | | | | |
| ApoE4 | | added | Glycocholic acid | | | |
| Surfactant concentration | | — | 0.030% | 0.050% | 0.10% | 0.20% |
| Solution A (w/v %) | | | | | | |
| Surfactant concentration | | — | 0.028% | 0.047% | 0.094% | 0.19% |
| Solution B (w/v %) | | | | | | |
| Comparison of | Average | 70% | 76% | 84% | 99% | 104% |
| measurement | value | | | | | |
| values (%) | CV | 12% | 7% | 4% | 6% | 4% |
| Lipid-rich/ | | | | | | |
| control sample | | | | | | |

TABLE 6

| | | Not | | | | |
|---|---|---|---|---|---|---|
| ApoE4 | | added | Taurocholic acid | | | |
| Surfactant concentration | | — | 0.030% | 0.050% | 0.10% | 0.20% |
| Solution A (w/v %) | | | | | | |
| Surfactant concentration | | — | 0.028% | 0.047% | 0.094% | 0.19% |
| Solution B (w/v %) | | | | | | |
| Comparison of | Average | 70% | 78% | 94% | 112% | 104% |
| measurement | value | | | | | |
| values (%) | CV | 12% | 8% | 1% | 6% | 8% |
| Lipid-rich/ | | | | | | |
| control sample | | | | | | |

As shown in Tables 5 and 6 above, it was found that, under all the concentration conditions (antibody-immobilized particle solution: 0.030 to 0.20%, reaction solution: 0.028 to 0.19%), cholic acid, glycocholic acid, or taurocholic acid could also suppress the measurement interference in the ApoE4 measurement by a lipid compared with the condition in which no surfactant had been added. In particular, it was found that the measurement interference in the ApoE4 measurement by a lipid could be further suppressed under the condition in which the concentrations of cholic acid, glycocholic acid, and taurocholic acid in the antibody-immobilized particle solution were 0.030 to 0.20%, 0.050 to 0.20%, and 0.050 to 0.20%, respectively, that is, the concentrations thereof in the reaction solution were 0.028 to 0.19%, 0.047 to 0.19%, and 0.047 to 0.19%, respectively. Also, it was found that, when cholic acid, glycocholic acid, and taurocholic acid were used, CV was sufficiently low under all the concentration conditions, and the inhibition of the ApoE measurement could be suppressed irrespective of the samples.

TABLE 7

| | | Not | | | | |
|---|---|---|---|---|---|---|
| ApoE4 | | added | Cholic acid | | | |
| Surfactant concentration | | — | 0.25% | 0.50% | 1.0% | 2.0% |
| Solution A (w/v %) | | | | | | |
| Surfactant concentration | | — | 0.23% | 0.47% | 0.94% | 1.9% |
| Solution B (w/v %) | | | | | | |
| Comparison of | Average | 59% | 103% | 98% | 107% | 104% |
| measurement | value | | | | | |
| values (%) | CV | 8% | 7% | 6% | 10% | 9% |
| Lipid-rich/ | | | | | | |
| control sample | | | | | | |

| | | Not | | | | |
|---|---|---|---|---|---|---|
| ApoE4 | | added | CHAPS | | | |
| Surfactant concentration | | — | 0.25% | 0.50% | 1.0% | 2.0% |
| Solution A (w/v %) | | | | | | |
| Surfactant concentration | | — | 0.23% | 0.47% | 0.94% | 1.9% |
| Solution B (w/v %) | | | | | | |
| Comparison of | Average | 59% | 96% | 96% | 95% | 95% |
| measurement | value | | | | | |
| values (%) | CV | 8% | 2% | 2% | 2% | 3% |
| Lipid-rich/ | | | | | | |
| control sample | | | | | | |

As shown in Table 7 above, it was found that, under all the concentration conditions (antibody-immobilized particle solution: 0.25 to 2.0%, reaction solution: 0.23 to 1.9%), cholic acid or CHAPS could also suppress the measurement interference in the ApoE4 measurement by a lipid compared with the condition in which no surfactant had been added. Also, it was found that, when cholic acid and CHAPS were used, CV was sufficiently low under all the concentration conditions, and the inhibition of the ApoE measurement could be suppressed irrespective of the samples.

TABLE 8

| | | Not | | | | |
|---|---|---|---|---|---|---|
| Pan-ApoE | | added | Deoxycholic acid | | | |
| Surfactant concentration | | — | 0.020% | 0.030% | 0.040% | 0.050% |
| Solution A (w/v %) | | | | | | |
| Surfactant concentration | | — | 0.019% | 0.029% | 0.038% | 0.048% |
| Solution C (w/v %) | | | | | | |
| Comparison of | Average | 67% | 76% | 76% | 82% | 85% |
| measurement | value | | | | | |
| values (%) | CV | 8% | 4% | 2% | 2% | 1% |
| Lipid-rich/ | | | | | | |
| control sample | | | | | | |

TABLE 9

| | | |
|---|---|---|
| | Not | |
| Pan-ApoE | added | Deoxycholic acid |

| Surfactant concentration | | | — | 0.20% | 0.30% |
|---|---|---|---|---|---|
| Solution A (w/v %) | | | | | |
| Surfactant concentration | | | — | 0.19% | 0.29% |
| Solution C (w/v %) | | | | | |
| | Comparison of | Average | 33% | 92% | 99% |
| | measurement | value | | | |
| | values (%) | CV | 16% | 0% | 4% |
| | Lipid-rich/ | | | | |
| | control sample | | | | |

As shown in Tables 8 and 9 above, it was found that, under all the concentration conditions (antibody-immobilized particle solution: 0.020 to 0.30%, reaction solution: 0.019 to 0.29%), deoxycholic acid could suppress the measurement interference in the Pan-ApoE measurement by a lipid compared with the condition in which no surfactant had been added. In particular, it was found that the measurement interference in the Pan-ApoE measurement by a lipid could be further suppressed under the condition in which the concentration of deoxycholic acid in the antibody-immobilized particle solution was 0.040 to 0.30%, that is, the concentration thereof in the reaction solution was 0.038 to 0.29%. Also, it was found that, when deoxycholic acid was used, CV was sufficiently low under all the concentration conditions, and the inhibition of the ApoE measurement could be suppressed irrespective of the samples.

TABLE 10

| | | Not | | | | |
|---|---|---|---|---|---|---|
| Pan-ApoE | | added | Cholic acid | | | |
| Surfactant concentration | | — | 0.030% | 0.050% | 0.10% | 0.20% |
| Solution A (w/v %) | | | | | | |
| Surfactant concentration | | — | 0.029% | 0.048% | 0.095% | 0.19% |
| Solution C (w/v %) | | | | | | |
| Comparison of | Average | 69% | 76% | 79% | 88% | 94% |
| measurement | value | | | | | |
| values (%) | CV | 0% | 5% | 5% | 2% | 2% |
| Lipid-rich/ | | | | | | |
| control sample | | | | | | |
| | | Not | | | | |
| Pan-ApoE | | added | Glycocholic acid | | | |
| Surfactant concentration | | — | 0.030% | 0.050% | 0.10% | 0.20% |
| Solution A (w/v %) | | | | | | |
| Surfactant concentration | | — | 0.029% | 0.048% | 0.095% | 0.19% |
| Solution C (w/v %) | | | | | | |
| Comparison of | Average | 69% | 75% | 79% | 83% | 93% |
| measurement | value | | | | | |
| values (%) | CV | 0% | 3% | 3% | 5% | 2% |
| Lipid-rich/ | | | | | | |
| control sample | | | | | | |

TABLE 11

| | | Not | | | | |
|---|---|---|---|---|---|---|
| Pan-ApoE | | added | Taurocholic acid | | | |
| Surfactant concentration | | — | 0.030% | 0.050% | 0.10% | 0.20% |
| Solution A (w/v %) | | | | | | |
| Surfactant concentration | | — | 0.029% | 0.048% | 0.095% | 0.19% |
| Solution C (w/v %) | | | | | | |
| Comparison of | Average | 69% | 76% | 83% | 89% | 89% |
| measurement | value | | | | | |
| values (%) | CV | 0% | 4% | 5% | 4% | 1% |
| Lipid-rich/ | | | | | | |
| control sample | | | | | | |

As shown in Tables 10 and 11 above, it was found that, under all the concentration conditions (antibody-immobilized particle solution: 0.030 to 0.20%, reaction solution: 0.029 to 0.19%), cholic acid, glycocholic acid, or taurocholic acid could also suppress the measurement interference in the Pan-ApoE measurement by a lipid compared with the condition in which no surfactant had been added. In particular, it was found that the measurement interference in the Pan-ApoE measurement by a lipid could be further suppressed under the condition in which the concentrations of cholic acid, glycocholic acid, and taurocholic acid in the antibody-immobilized particle solution were 0.10 to 0.20%, 0.10 to 0.20%, and 0.050 to 0.20%, respectively, that is, the concentrations thereof in the reaction solution were 0.095 to 0.19%, 0.095 to 0.19%, and 0.048 to 0.19%, respectively. Also, it was found that, when cholic acid, glycocholic acid, and taurocholic acid were used, CV was sufficiently low under all the concentration conditions, and the inhibition of the ApoE measurement could be suppressed irrespective of the samples.

TABLE 12

| | | Not | | | | |
|---|---|---|---|---|---|---|
| Pan-ApoE | | added | Cholic acid | | | |
| Surfactant concentration | | — | 0.25% | 0.50% | 1.0% | 2.0% |
| Solution A (w/v %) | | | | | | |
| Surfactant concentration | | — | 0.24% | 0.48% | 0.95% | 1.9% |
| Solution C (w/v %) | | | | | | |
| Comparison of | Average | 64% | 97% | 99% | 110% | 107% |
| measurement | value | | | | | |
| values (%) | CV | 13% | 3% | 2% | 5% | 5% |
| Lipid-rich/ | | | | | | |
| control sample | | | | | | |

| | | Not | | | | |
|---|---|---|---|---|---|---|
| Pan-ApoE | | added | CHAPS | | | |
| Surfactant concentration | | — | 0.25% | 0.50% | 1.0% | 2.0% |
| Solution A (w/v %) | | | | | | |
| Surfactant concentration | | — | 0.24% | 0.48% | 0.95% | 1.9% |
| Solution C (w/v %) | | | | | | |
| Comparison of | Average | 64% | 92% | 96% | 98% | 98% |
| measurement | value | | | | | |
| values (%) | CV | 13% | 3% | 4% | 2% | 1% |
| Lipid-rich/ | | | | | | |
| control sample | | | | | | |

As shown in Table 12 above, it was found that, under all the concentration conditions (antibody-immobilized particle solution: 0.25 to 2.0%, reaction solution: 0.24 to 1.9%), cholic acid and CHAPS could suppress the measurement interference in the Pan-ApoE measurement by a lipid compared with the condition in which no surfactant had been added. Also, it was found that, when cholic acid and CHAPS were used, CV was sufficiently low under all the concentration conditions, and the inhibition of the ApoE measurement could be suppressed irrespective of the samples.

It was found from the results above that the surfactants having a steroid skeleton other than CHAPS could also be used to suppress the measurement inhibition by a lipid in measurement of a wide range of ApoEs including ApoE4. It was assumed that the surfactants having a steroid skeleton exhibited an excellent ability to suppress the inhibition of ApoE measurement compared with the other surfactants because they could favorably act on a lipid bound to an ApoE due to the affinity of the steroid skeleton for a lipid to release the lipid from the ApoE. However, the present disclosure is not limited to the assumption above.

### Example 3

It was confirmed that adding a plurality of types of surfactants having a steroid skeleton to a blood sample made it possible to suppress inhibition of ApoE4 measurement and Pan-ApoE measurement by a lipid.

It was examined whether the ApoE4 measurement method described in Example 1 (2) above and the Pan-ApoE measurement method described in Example 1 (3) above could be used to suppress inhibition of measurement by a lipid in the same manner as in Example 1 above, except that a combination of the bile acid-based surfactants such as cholic acid, deoxycholic acid, glycocholic acid, and taurocholic acid was used. Tables 13 and 14 below show the ApoE4 measurement results, and Tables 15 and 16 below show the Pan-ApoE measurement results.

TABLE 13

| | | | Cholic acid | | Cholic acid | |
|---|---|---|---|---|---|---|
| | | | (Solution A: 0.050 w/v %) | | (Solution A: 0.10 w/v %) | |
| | | Not | Cholic acid | | Cholic acid | |
| ApoE4 | | added | (Solution B: 0.047 w/v %) | | (Solution B: 0.094 w/v %) | |
| Deoxycholic acid | | Not | 0.020% | 0.040% | 0.00% | 0.020% |
| concentration | | added | | | | |
| Solution A (w/v %) | | | | | | |
| Deoxycholic acid | | Not | 0.019% | 0.038% | 0.00% | 0.019% |
| concentration | | added | | | | |
| Solution B (w/v %) | | | | | | |
| Comparison of | Average | 68% | 98% | 107% | 101% | 112% |
| measurement | value | | | | | |
| values (%) | CV | 9% | 4% | 14% | 4% | 12% |
| Lipid-rich/ | | | | | | |
| control sample | | | | | | |
| | | | Glycocholic acid | | Glycocholic acid | |
| | | | (Solution A: 0.050 w/v %) | | (Solution A: 0.10 w/v %) | |
| | | Not | Glycocholic acid | | Glycocholic acid | |
| ApoE4 | | added | (Solution B: 0.047 w/v %) | | (Solution B: 0.094 w/v %) | |
| Deoxycholic acid | | Not | 0.020% | 0.040% | 0.00% | 0.020% |
| concentration | | added | | | | |
| Solution A (w/v %) | | | | | | |
| Deoxycholic acid | | Not | 0.019% | 0.038% | 0.00% | 0.019% |
| concentration | | added | | | | |
| Solution B (w/v %) | | | | | | |
| Comparison of | Average | 68% | 88% | 107% | 99% | 108% |
| measurement | value | | | | | |
| values (%) | CV | 9% | 1% | 13% | 2% | 8% |
| Lipid-rich/ | | | | | | |
| control sample | | | | | | |

TABLE 14

| | | | Taurocholic acid | | | Taurocholic acid | |
|---|---|---|---|---|---|---|---|
| | | | (Solution A: 0.025 w/v %) | | | (Solution A: 0.050 w/v %) | |
| | | Not | Taurocholic acid | | | Taurocholic acid | |
| ApoE4 | | added | (Solution B: 0.023 w/v %) | | | (Solution B: 0.047 w/v %) | |
| Deoxycholic acid | | Not | 0.00% | 0.020% | 0.040% | 0.00% | 0.020% |
| concentration | | added | | | | | |
| Solution A (w/v %) | | | | | | | |
| Deoxycholic acid | | Not | 0.00% | 0.019% | 0.038% | 0.00% | 0.019% |
| concentration | | added | | | | | |
| Solution B (w/v %) | | | | | | | |
| Comparison of | Average | 68% | 78% | 88% | 99% | 85% | 99% |
| measurement | value | | | | | | |
| values (%) | CV | 9% | 5% | 4% | 7% | 0% | 4% |
| Lipid-rich/ | | | | | | | |
| control sample | | | | | | | |

As shown in Tables 13 and 14 above, it was found that the combination of deoxycholic acid and any of cholic acid, glycocholic acid, and taurocholic acid could suppress the measurement interference in the ApoE4 measurement by a lipid compared with the condition in which no surfactant had been added. Also, it was found that, when any of the combinations was used, CV was sufficiently low under all the concentration conditions, and the inhibition of the ApoE measurement could be suppressed irrespective of the samples.

TABLE 15

| | | | Cholic acid | | | Cholic acid |
|---|---|---|---|---|---|---|
| | | | (Solution A: 0.20 w/v %) | | | (Solution A: 0.30 w/v %) |
| | | Not | Cholic acid | | | Cholic acid |
| Pan-ApoE | | added | (Solution C: 0.19 w/v %) | | | (Solution C: 0.29 w/v %) |
| Deoxycholic acid | | Not | 0.00% | 0.040% | 0.070% | 0.00% |
| concentration | | added | | | | |
| Solution A (w/v %) | | | | | | |
| Deoxycholic acid | | Not | 0.00% | 0.038% | 0.067% | 0.00% |
| concentration | | added | | | | |
| Solution C (w/v %) | | | | | | |
| Comparison of | Average | 73% | 97% | 100% | 100% | 102% |
| measurement | value | | | | | |
| values (%) | CV | 8% | 0% | 1% | 3% | 1% |
| Lipid-rich/ | | | | | | |
| control sample | | | | | | |
| | | | Glycocholic acid | | | Glycocholic acid |
| | | | (Solution A: 0.20 w/v %) | | | (Solution A: 0.30 w/v %) |
| | | Not | Glycocholic acid | | | Glycocholic acid |
| Pan-ApoE | | added | (Solution C: 0.19 w/v %) | | | (Solution C: 0.29 w/v %) |
| Deoxycholic acid | | Not | 0.00% | 0.040% | 0.070% | 0.00% |
| concentration | | added | | | | |
| Solution A (w/v %) | | | | | | |
| Deoxycholic acid | | Not | 0.00% | 0.038% | 0.067% | 0.00% |
| concentration | | added | | | | |
| Solution C (w/v %) | | | | | | |
| Comparison of | Average | 73% | 87% | 100% | 100% | 95% |
| measurement | value | | | | | |
| values (%) | CV | 8% | 4% | 3% | 2% | 5% |
| Lipid-rich/ | | | | | | |
| control sample | | | | | | |

TABLE 16

| | | | Taurocholic acid | | | Taurocholic acid |
|---|---|---|---|---|---|---|
| | | | (Solution A: 0.20 w/v %) | | | (Solution A: 0.30 w/v %) |
| | | Not | Taurocholic acid | | | Taurocholic acid |
| Pan-ApoE | | added | (Solution C: 0.19 w/v %) | | | (Solution C: 0.29 w/v %) |
| Deoxycholic acid | | Not | 0.00% | 0.040% | 0.070% | 0.00% |
| concentration | | added | | | | |
| Solution A (w/v %) | | | | | | |
| Deoxycholic acid | | Not | 0.00% | 0.038% | 0.067% | 0.00% |
| concentration | | added | | | | |
| Solution C (w/v %) | | | | | | |
| Comparison of | Average | 73% | 94% | 99% | 104% | 100% |
| measurement | value | | | | | |
| values (%) | CV | 8% | 3% | 2% | 3% | 6% |
| Lipid-rich/ | | | | | | |
| control sample | | | | | | |

As shown in Tables 15 and 16 above, it was found that the combination of deoxycholic acid and cholic acid, the combination of deoxycholic acid and glycocholic acid, or the combination of deoxycholic acid and taurocholic acid could suppress the measurement inhibition by a lipid. Also, it was found that, when any of the combinations was used, CV was sufficiently low under all the concentration conditions, and the inhibition of the ApoE measurement could be suppressed irrespective of the samples.

It was found from the results above that using surfactants having a steroid skeleton such as CHAPS and bile acid-based surfactants independently or in combination makes it possible to effectively suppress inhibition of Pan-ApoE measurement and ApoE4 measurement even in lipid-rich blood specimens (e.g., blood specimens from patients suffering from hyperlipemia) containing neutral fat, cholesterol, and the like in large amounts.

As described above, the present disclosure has been described with reference to the embodiments and the examples, but the present disclosure is not limited to the above-described embodiments and examples. Various modifications that can be understood by a person skilled in the art can be made in the configurations and details of the present disclosure without departing from the scope of the present disclosure.

### <Supplementary Notes>

Some or all of the embodiments and the examples given above can be described as in the following supplementary notes, but the scope of the present disclosure is not limited thereto.

### <Composition for Use to Suppress Inhibition of ApoE Measurement by Lipid

### (Supplementary Note 1)

A composition for use to suppress inhibition of apolipoprotein E (ApoE) measurement by a lipid, the composition including a surfactant having a steroid skeleton.

### (Supplementary Note 2)

The composition according to Supplementary Note 1, wherein the surfactant is bile acid or a derivative thereof, or a salt thereof.

### (Supplementary Note 3)

The composition according to Supplementary Note 1 or 2, wherein the surfactant has a steroid skeleton that does not have a hydroxyl group at position 7.

### (Supplementary Note 4)

The composition according to any one of Supplementary Notes 1 to 3, wherein the surfactant is cholic acid, deoxycholic acid, glycocholic acid, or taurocholic acid, or a salt thereof.

### (Supplementary Note 5)

The composition according to any one of Supplementary Notes 1 to 4, wherein the ApoE is an ApoE4 and/or total ApoE.

### <Kit for Use to Measure ApoE>

### (Supplementary Note 6)

A kit for use to measure an apolipoprotein E (ApoE), including:
a surfactant having a steroid skeleton; and
an apolipoprotein E (ApoE) measurement reagent.

### (Supplementary Note 7)

The kit according to Supplementary Note 6, wherein the surfactant is bile acid or a derivative thereof, or a salt thereof.

### (Supplementary Note 8)

The kit according to Supplementary Note 6 or 7, wherein the surfactant has a steroid skeleton that does not have a hydroxyl group at position 7.

### (Supplementary Note 9)

The kit according to Supplementary Note 7, wherein the surfactant is cholic acid, deoxycholic acid, glycocholic acid, or taurocholic acid, or a salt thereof.

### (Supplementary Note 10)

The kit according to any one of Supplementary Notes 6 to 9, wherein the ApoE measurement reagent contains a first binding molecule for an ApoE.

### (Supplementary Note 11)

The kit according to any one of Supplementary Notes 6 to 10, wherein the ApoE measurement reagent contains a carrier supporting a first binding molecule for an ApoE.

### (Supplementary Note 12)

The kit according to Supplementary Note 11, wherein the carrier is a magnetic particle.

### (Supplementary Note 13)

The kit according to any one of Supplementary Notes 10 to 12, wherein the ApoE measurement reagent has a label and contains a second binding molecule for the ApoE.

### (Supplementary Note 14)

The kit according to Supplementary Note 13,
wherein the label is an enzyme, and
the kit includes a substrate of the enzyme.

### (Supplementary Note 15)

The kit according to any one of Supplementary Notes 6 to 14, wherein the ApoE is an ApoE4 and/or total ApoE.

### <Method for Suppressing Inhibition of ApoE Measurement by Lipid>

### (Supplementary Note 16)

A method for suppressing inhibition of apolipoprotein E (ApoE) measurement by a lipid, including a coexistence step of allowing a target sample to coexist with a surfactant having a steroid skeleton.

### (Supplementary Note 17)

The suppression method according to Supplementary Note 16, wherein the sample is brought into contact with a solution containing the surfactant in the coexistence step.

### (Supplementary Note 18)

The suppression method according to Supplementary Note 16 or 17, wherein the surfactant is bile acid or a derivative thereof, or a salt thereof.

### (Supplementary Note 19)

The suppression method according to any one of Supplementary Notes 16 to 18, wherein the surfactant has a steroid skeleton that does not have a hydroxyl group at position 7.

### (Supplementary Note 20)

The suppression method according to Supplementary Note 18, wherein the surfactant is cholic acid, deoxycholic acid, glycocholic acid, or taurocholic acid, or a salt thereof.

### (Supplementary Note 21)

The suppression method according to any one of Supplementary Notes 16 to 20, wherein the ApoE is an ApoE4 and/or total ApoE.

### (Supplementary Note 22)

The suppression method according to any one of Supplementary Notes 16 to 21, wherein the sample includes a biological sample.

### (Supplementary Note 23)

The suppression method according to Supplementary Note 22, wherein the biological sample is a blood sample.

### <Method for Measuring ApoE

### (Supplementary Note 24)

A method for measuring an ApoE, including a measurement step of measuring an ApoE in a target sample by bringing the sample into contact with an ApoE measurement reagent in the presence of a surfactant having a steroid skeleton.

### (Supplementary Note 25)

The measurement method according to Supplementary Note 24,
wherein the measurement step includes:
   a first complex formation step of forming a first complex composed of the ApoE in the sample and a first binding molecule for an ApoE by bringing the sample into contact with the first binding molecule in the presence of the surfactant having a steroid skeleton; and
   a complex measurement step of measuring the ApoE in the sample by measuring the first complex.

### (Supplementary Note 26)

The measurement method according to Supplementary Note 24,
wherein the measurement step includes:
   a first complex formation step of forming a first complex composed of the ApoE in the sample and a first binding molecule for an ApoE by bringing the sample into contact with the first binding molecule in the presence of the surfactant having a steroid skeleton;
   a second complex formation step of forming a second complex composed of the first complex and a second binding molecule for an ApoE by bringing the first complex into contact with the second binding molecule; and
   a complex measurement step of measuring the ApoE in the sample by measuring the second complex.

### (Supplementary Note 27)

The measurement method according to Supplementary Note 26, wherein the first binding molecule is supported by a carrier.

### (Supplementary Note 28)

The measurement method according to Supplementary Note 26 or 27,
wherein the second complex has a label, and
the ApoE in the sample is measured by measuring the label in the second complex in the complex measurement step.

### (Supplementary Note 29)

The measurement method according to any one of Supplementary Notes 24 to 28, including a coexistence step of bringing the sample into contact with a solution containing the surfactant prior to the measurement step.

### (Supplementary Note 30)

The measurement method according to any one of Supplementary Notes 24 to 29, wherein the surfactant is bile acid or a derivative thereof, or a salt thereof.

### (Supplementary Note 31)

The measurement method according to any one of Supplementary Notes 24 to 30, wherein the surfactant has a steroid skeleton that does not have a hydroxyl group at position 7.

### (Supplementary Note 32)

The measurement method according to Supplementary Note 30, wherein the surfactant is cholic acid, deoxycholic acid, glycocholic acid, or taurocholic acid, or a salt thereof.

### (Supplementary Note 33)

The measurement method according to any one of Supplementary Notes 24 to 32, wherein the ApoE is an ApoE4 and/or total ApoE.

### (Supplementary Note 34)

The measurement method according to any one of Supplementary Notes 24 to 33, wherein the sample includes a biological sample.

### (Supplementary Note 35)

The measurement method according to Supplementary Note 34, wherein the biological sample is a blood sample.

### <Method for Testing Likelihood of ARIA>

### (Supplementary Note 36)

A method for testing a likelihood of amyloid-related imaging abnormalities when a disease-modifying drug for Alzheimer's disease (AD) is administered to a target, the method including
a measurement step of measuring an ApoE in a sample from the target by bringing the sample into contact with an ApoE measurement reagent in the presence of a surfactant having a steroid skeleton.

### (Supplementary Note 37)

The test method according to Supplementary Note 36, including an evaluation step of evaluating a likelihood that the ARIA occurs based on genotype analysis results of the ApoE obtained in the measurement step.

### (Supplementary Note 38)

The test method according to Supplementary Note 37, including an administration step of administering the disease-modifying drug for AD based on evaluation results obtained in the evaluation step.

### (Supplementary Note 39)

The test method according to any one of Supplementary Notes 36 to 38, wherein the disease-modifying drug for Alzheimer's diseases is an anti-amyloid β antibody.

### (Supplementary Note 40)

The test method according to any one of Supplementary Notes 36 to 39,
wherein the measurement step includes:
   a first complex formation step of forming a first complex composed of the ApoE in the sample and a first binding molecule for an ApoE by bringing the sample into contact with the first binding molecule in the presence of the surfactant having a steroid skeleton; and
   a complex measurement step of measuring the ApoE in the sample by measuring the first complex.

### (Supplementary Note 41)

The test method according to any one of Supplementary Notes 36 to 39,
wherein the measurement step includes:
   a first complex formation step of forming a first complex composed of the ApoE in the sample and a first binding molecule for an ApoE by bringing the sample into contact with the first binding molecule in the presence of the surfactant having a steroid skeleton;
   a second complex formation step of forming a second complex composed of the first complex and a second binding molecule for an ApoE by bringing the first complex into contact with the second binding molecule; and
   a complex measurement step of measuring the ApoE in the sample by measuring the second complex.

### (Supplementary Note 42)

The test method according to Supplementary Note 41, wherein the first binding molecule is supported by a carrier.

### (Supplementary Note 43)

The test method according to Supplementary Note 41 or 42,
wherein the second complex has a label, and
the ApoE in the sample is measured by measuring the label in the second complex in the complex measurement step.

### (Supplementary Note 44)

The test method according to any one of Supplementary Notes 36 to 43, including a coexistence step of bringing the sample into contact with a solution containing the surfactant prior to the measurement step.

### (Supplementary Note 45)

The test method according to any one of Supplementary Notes 36 to 44, wherein the surfactant is bile acid or a derivative thereof, or a salt thereof.

### (Supplementary Note 46)

The test method according to any one of Supplementary Notes 36 to 45, wherein the surfactant has a steroid skeleton that does not have a hydroxyl group at position 7.

### (Supplementary Note 47)

The test method according to Supplementary Note 45, wherein the surfactant is cholic acid, deoxycholic acid, glycocholic acid, or taurocholic acid, or a salt thereof.

### (Supplementary Note 48)

The test method according to any one of Supplementary Notes 36 to 47, wherein the ApoE is an ApoE4 and/or total ApoE.

### (Supplementary Note 49)

The test method according to any one of Supplementary Notes 36 to 48, wherein the sample includes a biological sample.

### (Supplementary Note 50)

The test method according to Supplementary Note 49, wherein the biological sample is a blood sample.

### <Use>

### (Supplementary Note 51)

Use of the composition according to any one of Supplementary Notes 1 to 5 for use to suppress inhibition of apolipoprotein E (ApoE) measurement by a lipid.

### (Supplementary Note 52)

Use of the kit according to any one of Supplementary Notes 6 to 15 for use to measure an ApoE while suppressing an inhibitory effect of a lipid on ApoE measurement.

### INDUSTRIAL APPLICABILITY

As described above, with the present disclosure, it is possible to suppress an inhibitory effect of a lipid on the ApoE measurement. Accordingly, the present disclosure is very useful in the fields of, for example, inspection and the like.

1. A kit for use to measure an apolipoprotein E (ApoE), comprising:
a surfactant having a steroid skeleton; and
an ApoE measurement reagent.

2. The kit according to claim 1, wherein
the surfactant is bile acid or a derivative thereof, or a salt thereof.

3. The kit according to claim 2, wherein
the surfactant is cholic acid, deoxycholic acid, glycocholic acid, or taurocholic acid, or a salt thereof.

4. The kit according to claim 1, wherein
the ApoE measurement reagent comprises a first binding molecule for an ApoE.

5. The kit according to claim 1, wherein
the ApoE measurement reagent comprises a carrier comprising a first binding molecule for an ApoE.

6. The kit according to claim 5, wherein
the ApoE measurement reagent comprises a label and contains a second binding molecule for the ApoE.

7. The kit according to claim 6, wherein
the label is an enzyme, and
the kit comprises a substrate of the enzyme.

8. The kit according to claim 1, wherein
the ApoE is an ApoE4 and/or total ApoE.

9. A method for measuring an ApoE, comprising
measuring an ApoE in a target sample by bringing the sample into contact with an ApoE measurement reagent in the presence of a surfactant having a steroid skeleton.

10. The measurement method according to claim 9,
wherein the measuring comprises:
forming a first complex composed of the ApoE in the sample and a first binding molecule for an ApoE by bringing the sample into contact with the first binding molecule in the presence of the surfactant having a steroid skeleton;
forming a second complex composed of the first complex and a second binding molecule for an ApoE by bringing the first complex into contact with the second binding molecule; and
measuring the ApoE in the sample by measuring the second complex.

11. The measurement method according to claim 10, wherein
the first binding molecule comprises a carrier.

12. The measurement method according to claim 11, wherein
the second complex comprises a label, and
the measuring of the complex is the measuring of the ApoE in the sample by measuring the label in the second complex.

13. The measurement method according to claim 9, wherein
the surfactant is bile acid or a derivative thereof, or a salt thereof.

14. The measurement method according to claim 13, wherein
the surfactant is cholic acid, deoxycholic acid, glycocholic acid, or taurocholic acid, or a salt thereof.

15. The measurement method according to claim 9, wherein
an inhibition of measuring the ApoE by lipids is suppressed compared with the measuring of the sample without the surfactant.

16. The measurement method according to claim 9, wherein
the ApoE is an ApoE4 and/or total ApoE.

17. The measurement method according to claim 9, wherein
the sample comprises a biological sample.

18. The measurement method according to claim 17, wherein
the biological sample is a blood sample.
